SEQUENCE LISTING (36 sequences)

SEQ ID NO: 1 - protein, 38 aa, ARTIFICIAL
  TALENs binding domain, left
NINGNIHDNINGNGNGNGNIHDNIHDNIHDNINGNING

SEQ ID NO: 2 - protein, 34 aa, ARTIFICIAL
  TALENs binding domain, right.
NINNNNNGNGHDNINNNNHDHDNINGNGNINING

SEQ ID NO: 3 - protein, 32 aa, ARTIFICIAL
  TALENs binding domain, left.
NNHDHDHDHDHDHDHDNIHDNIHDNINNHDNG

SEQ ID NO: 4 - protein, 32 aa, ARTIFICIAL
  TALENs binding domain, right.
NINGNINNHDHDNGHDNINNNNNNNGNIHDNG

SEQ ID NO: 5 - protein, 36 aa, ARTIFICIAL
  TALENs binding domain, left.
NNNNNINIHDNINGNNHDNINGNGHDNINIHDNING

SEQ ID NO: 6 - protein, 40 aa, ARTIFICIAL
  TALENs binding domain, right.
HDHDNINNNGNINNNGNGNGHDNGHDNGNINGNNHDHDNG

SEQ ID NO: 7 - protein, 36 aa, ARTIFICIAL
  TALENs binding domain, left.
NINGNINGNNNININIHDNGNNNIHDNINNNGNING

SEQ ID NO: 8 - protein, 38 aa, ARTIFICIAL
  TALENs binding domain, right.
HDHDHDNININGHDNGNNNINNNGNGHDNGNNNNHDNG

SEQ ID NO: 9 - DNA, 23 bp, ARTIFICIAL
  PCR PRIMERS, FORWARD
gctcctggccatctctttggtca

SEQ ID NO: 10 - DNA, 23 bp, ARTIFICIAL
  PCR PRIMERS REVERSE
tgcctgcctgcttgcatctctca

SEQ ID NO: 11 - DNA, 23 bp, ARTIFICIAL
  PCR PRIMER, FORWARD
gcaataacactgacccgaccgtg

SEQ ID NO: 12 - DNA, 24 bp, ARTIFICIAL
  PCR PRIMER, REVERSE
gcaggtgtcagccctttaggagct

SEQ ID NO: 13 - DNA, 25 bp, ARTIFICIAL
  PCR PRIMER, FORWARD
ggaatatgggcatgtgttgtcagtc

SEQ ID NO: 14 - DNA, 25 bp, ARTIFICIAL
  PCR PRIMER, REVERSE
tgcagtatacttcatccacgaggca

SEQ ID NO: 15 - protein, 32 aa, ARTIFICIAL
  Left Monomer RVD sequence
NNNINIHDNNHDNGNGNGHDNINGNGNNNGNN

SEQ ID NO: 16 - protein, 32 aa, ARTIFICIAL
  Right Monomer RVD sequence
NINNNINNHDHDNIHDNGNGNGNGHDNGHDHD

SEQ ID NO: 17 - DNA, 48 bp, ARTIFICIAL
  SENSE STRAND BINDING SEQUENCE WITH SPACER
gaacgctttcattgtgtggtctcgtgatcaaaggagaaaagtggctct

SEQ ID NO: 18 - protein, 32 aa, ARTIFICIAL
  Left Monomer RVD seqeunce
NINNNINNNINIHDHDHDNGHDNINININGNN

SEQ ID NO: 19 - protein, 34 aa, ARTIFICIAL
  Right Monomer RVD sequence
NNHDNINGHDHDHDNINNHDHDNIHDNGNGNNHD

SEQ ID NO: 20 - DNA, 49 bp, ARTIFICIAL
  Sense strand binding sequence with spacer
agagaaccctcaaatgcaaaactcagagatcagcaagtggctgggatgc

SEQ ID NO: 21 - protein, 32 aa, ARTIFICIAL
  Left Monomer RVD seqeunce
HDNINNHDNININNNGNNNNHDNGNNNNNNNI

SEQ ID NO: 22 - protein, 30 aa, ARTIFICIAL
  Right Monomer RVD sequence
NGNGNGHDNNNNHDNGNGHDNGNNNGNINI

SEQ ID NO: 23 - DNA, 47 bp, ARTIFICIAL
  Sense strand binding sequence with spacer
cagcaagtggctgggatgcaagtggaaaatgcttacagaagccgaaa

SEQ ID NO: 24 - protein, 34 aa, ARTIFICIAL
  Left Monomer RVD Sequence
HDHDNNNGNNNGNINNHDHDNININGNNNGNGNI

SEQ ID NO: 25 - protein, 36 aa, ARTIFICIAL
  Right Monomer RVD Sequence
HDNIHDNINNHDNINNHDNGNNNNNIHDNININNHD

SEQ ID NO: 26 - DNA, 51 bp, ARTIFICIAL
  Sense strand binding sequence with spacer
ccgtgtagccaatgttaccttattgtggcccaggcttgtccagctgctgtg

SEQ ID NO: 27 - protein, 34 aa, ARTIFICIAL
  Left Monomer RVD Sequence
NGNNNGNNNNHDHDHDNINNNNHDNGNGNNNGHD

SEQ ID NO: 28 - protein, 32 aa, ARTIFICIAL
  Right Monomer RVD Sequence
NGHDNIHDNGHDHDNGNNHDNININININNNN

SEQ ID NO: 29 - DNA, 49 bp, ARTIFICIAL
  Sense strand binding sequence with spacer
tgtggcccaggcttgtccagctgctgtgatgctccttttgcaggagtga

SEQ ID NO: 30 - protein, 36 aa, ARTIFICIAL
  Left Monomer RVD Sequence
HDNGHDNGNNNGNNHDHDNGHDHDNGHDNINININN

SEQ ID NO: 31 - protein, 36 aa, ARTIFICIAL
  Right Monomer RVD Sequence
NNNNNININININNNNHDNGNGNIHDNINNNINGNN

SEQ ID NO: 32 - DNA, 52 bp, ARTIFICIAL
  Sense strand binding sequence with spacer
ctctgtgcctcctcaaagaatgggcgcttttcagcatctgtaagccttttcc

SEQ ID NO: 33 - protein, 34 aa, ARTIFICIAL
  Left Monomer RVD Sequence
NGNNNIHDNNNGNNNNNGHDHDNGNNNNHDNGNN

SEQ ID NO: 34 - protein, 32 aa, ARTIFICIAL
  Right Monomer RVD Sequence
HDNINNNGNNNGNNNINININNNNNNNNNINN

SEQ ID NO: 35 - DNA, 49 bp, ARTIFICIAL
  Sense strand binding sequence with spacer
tgacgtggtcctggctgctctccctaacatgttctcccctttcacactg

SEQ ID NO: 36 - DNA, 90 bp, ARTIFICIAL
  Single-stranded DNA oligonucleotide template
cgtgtcaagcgacccatgaacgctttcattgtgtggtctcgttaagcttgatcaaaggagaaaagtggctctagagaaccctcaaatgca

(19) United States
(12) Patent Application Publication
(10) Pub. No.: US 2017/0099813 A1
(43) Pub. Date: Apr. 13, 2017

(54) METHOD OF GENERATING STERILE TERMINAL SIRES IN LIVESTOCK AND ANIMALS PRODUCED THEREBY

(71) Applicant: Recombinetics, Inc., Saint Paul, MN (US)

(72) Inventors: Scott C. Fahrenkrug, Minneapolis, MN (US); Tad S. Sonstegard, Centreville, MD (US); Daniel F. Carlson, Woodbury, MN (US)

(21) Appl. No.: 15/288,045

(22) Filed: Oct. 7, 2016

Related U.S. Application Data
(60) Provisional application No. 62/238,399, filed on Oct. 7, 2015.
(60) Provisional application No. 62/269,668, filed on Dec. 18, 2015.

(51) Int. Cl. A01K 67/027; C12N 15/113

(52) U.S. Cl. CPC A01K 67/0278; C12N 15/113; A01K 67/0271; C12N 2320/34; C12N 2310/10; A01K 2207/05; A01K 2207/12; A01K 2217/15; A01K 2227/10

(57) ABSTRACT

Disclosed herein are animals that are modified genetically to express one or more introduced sry alleles or to have a knockout of an existing sry allele. In various embodiments, the sry allele is inserted in at least one X chromosome of an XX resulting in an animal having two X chromosomes and appearing phenotypically male and sterile. In some embodiments the animal is modified to express multiple copies of sry inserted throughout its genome including the X chromosomes and the autosomes. In still other embodiments, the sry allele is inserted in multiple allosome sites. In these embodiments, XX individuals will be sterile and phenotypically male. XY individuals will be male and fertile but offspring will have an opportunity to inherit multiple sry alleles. In this embodiment, progeny of a male carrier will be sterile and phenotypically male if they are genotypically XX and express sry. Male progeny will be fertile and normal but will carry a hereditable copy of sry in their allosomes.

### CROSS REFERENCE TO RELATED APPLICATIONS

This application claims priority to U.S. Provisional Application Nos. 62/238,399 filed Oct. 7, 2015 and 62/269,668 filed Dec. 18, 2015 each of which is incorporated by reference in their entirety for all purposes.

### FIELD OF THE INVENTION

The invention is directed to livestock modified to express or knockout sry.

In livestock and other animal management and population control, the control of gender and reproduction affords the ability to determine the breeding potential, secondary characteristics and its ability of animals compete and reproduce. Many agricultural uses for domestic animals are gender specific such male steers for meat, because they are larger; cows for use in dairy, because they produce milk or the decision to castrate males because they are more docile. In addition, in managing wild animals, the ability to provide determine the sex of the offspring or a sterile gender provides a way to manipulate the population and mating success.

Sry is an intronless gene on the Y chromosome that has been identified as the “testis determining factor” in animals of the subclass “theria” (mammals and marsupials) animals. Mutations and duplication of this gene is responsible for a range of genotypic and phenotypic, sex-related effects. However, while the gene is initiates extremely critical process in therian development, it is little conserved among the subclass with the exception of a single conserved sequence for DNA binding.

In agriculture, a mix of culling, castrating and managed breeding are used for population control and maintaining livestock quality. In a wild setting, feral and wild animals have been sterilized and released back into the wild to provide sham-matings for population control. However, the process of castration for cattle, sheep, horses, pigs etc. requires considerable time, effort and money while the attempted management of feral and wild animal populations is even more problematic and requires huge resources.

Therefore, the ability to modify gender and fertility would be useful for both livestock animals and therian population control in general.

### SUMMARY OF THE INVENTION

The present disclosure provides therian or other livestock animal and methods to provide them that are genomically modified to provide a sterile phenotype. Further, the disclosure provides that the sterile livestock animal can be phenotypically either male or female. In addition the disclosure provides therian animals that are phenotypically and genotypically male but that also carry a hereditable, autosomal copy of sry. When inherited by a female offspring, the animal will be phenotypically male and sterile. Such animals are beneficial in providing an animal that is phenotypically a desired gender but sterile. Therian animals include marsupials, including kangaroos and wallaby's as well as livestock, such as, for example, cattle, horses, pigs, goats and the like which are for agricultural purposes often castrated such as steer, geldings, goats, sheep and the like. In addition, population control of other animals including, for example feral dogs, cats, pigs etc. and pests such as, for example, rats, mice and the like are also provided.

Therefore, in one exemplary embodiment, the disclosure teaches a therian animal comprising one or more introduced sry alleles. In some exemplary embodiments, the animal as an X and a Y chromosome and is phenotypically male. In some exemplary embodiments, the animal has two X chromosomes and is phenotypically male. In these exemplary embodiments, the animal is sterile. In various embodiments, the sry allele is introduced on one X chromosome. In other exemplary embodiments, the sry allele is introduced on both X chromosomes. In yet other exemplary embodiments, the sry allele is introduced on one or more autosomes. In these embodiments, the sry allele is hereditable when the animal has an X and a Y chromosome. In various embodiments, the animal is a livestock animal. In other embodiments the animal is a feral animal.

In yet other exemplary embodiments, the disclosure provides a therian animal comprising a genomic modification of an HMG box of the sry gene. In these exemplary embodiments, the animal has an XY genotype and is phenotypically female. In various embodiments, the animal is sterile. In yet other exemplary embodiments, the animal is a livestock animal. In various exemplary embodiments, the modification is an insertion or a deletion. In some embodiments, the modification results in a break in protein synthesis. In other exemplary embodiments, the modification results in a sry protein that fails to bind to its target DNA site or to initiate target synthesis. In various exemplary embodiments, the genomic modification is made by precision gene editing.

In still other exemplary embodiments, the disclosure teaches methods to provide an animal that is genotypically XX but phenotypically male. In various embodiments, the animal is sterile. In some exemplary embodiments, the method comprises editing the genome to include one or more sry alleles into the genome of a therian animal. In various exemplary embodiments, the sry transgene is inserted by nonmeiotic gene editing using zinc finger nuclease, meganuclease, TALENs or CRISPR/CAS technology. In some exemplary embodiments, the sry gene is under the control of its native promoter. In other exemplary embodiments, the sry gene is under control of an inducible promoter. In still other exemplary embodiments, the sry gene is under the control of a constitutive promoter. In some exemplary embodiments, the sry gene is inserted into at least one allosome of the animal. In other exemplary embodiments the sry gene is inserted into both allosomes of the animal. In yet other exemplary embodiments, the sry gene is inserted into one or more autosomes. In various exemplary embodiments, the genome editing is accomplished by nonmeiotic introgression. In some embodiments, the therian animal is a livestock animal. In other exemplary embodiments, the animal is a feral animal.

In yet other exemplary embodiments, the disclosure teaches methods to provide an animal that is genotypically XY and phenotypically male but carries at least one introduced sry allele in a chromosome other than the Y chromosome. In various exemplary embodiments, the introduced sry allele is present on at least one autosome. In some embodiments, the non-native sry allele is present in multiple copies. In some exemplary embodiments, the non-native sry allele is inserted in multiple chromosomes. In some exemplary embodiments, the method comprises editing the genome to include one or more sry alleles in the genome of a livestock animal. In other exemplary embodiments, the sry allele is introduced into a feral animal. In various exemplary embodiments, the sry allele is inserted by nonmeiotic gene editing using zinc finger nuclease, meganuclease, TALENs or CRISPR/CAS technology. In exemplary embodiments, the genome editing is accomplished by nonmeiotic introgression. In some exemplary embodiments, the sry allele is under the control of its native promoter. In other exemplary embodiments, the sry allele is under control of an inducible promoter. In still other exemplary embodiments, the sry allele is under the control of a constitutive promoter. In various exemplary embodiments, the sry allele is hereditable.

In another exemplary embodiment, the disclosure teaches methods to provide a livestock animal that is genotypically XY but is phenotypically female. In various embodiments, the phenotypically female animal is sterile. In this and other exemplary embodiments, the animal is modified at its sry gene. In some exemplary embodiments, the modification is in the HMG box. In various exemplary embodiments, the modification is an insertion or a deletion. In some exemplary embodiments, the deletion is a deletion of a single nucleotide. In other exemplary embodiments, the deletion is a deletion of up to 5 nucleotides. In still other exemplary embodiments, the deletion is a deletion of 10 nucleotides or more. In yet other embodiments, the mutation is an insertion. In various exemplary embodiments, the insertion is a frameshift insertion. In yet other exemplary embodiments, the insertion is a nonsense insertion. In these exemplary embodiments, the modification results in a break in protein synthesis. In other exemplary embodiments, the modification results in an inability of the sry protein to bind to its DNA target. In these and other exemplary embodiments, the genetic modification is made by precision gene editing using zinc finger nuclease, meganuclease, TALENs or CRISPR/CAS technology. In various exemplary embodiments, the genetic modification is made by nonmeiotic introgression

These and other features and advantages of the disclosure will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be apparent from the description, as set forth hereinafter.

### BRIEF DESCRIPTION OF THE DRAWINGS

Various exemplary embodiments of the compositions and methods according to the invention will be described in detail, with reference to the following figures wherein:

FIG. 1 is a diagram of the homology of the mouse sry gene with human sry;

FIG. 2 is a diagram of the homology of human sry with other species of therians illustrating the conserved nature of the HMG box compared to the rest of the gene;

FIG. 3 is a diagram of one strategy for the process of nonmeiotic introgression using TALENs;

FIGS. 4A-4C Production of ssODN-mediated knockouts of ssSRY. 4a) Three different TALEN pairs were designed to target ssSRY in Exon 1, and their efficiency was measured with a Surveyor Nuclease assay. 4b) RFLP assay on a population of cells transfected with the ssODN and the TALEN pair ssSRY1.1. 4c) Single-cell derived clones were genotyped via RFLP assay with HindIII.

FIG. 5 is a diagram of one strategy of nonmeiotic introgression using transposons;

FIG. 6 illustrates the use of multiple transposon systems in pig cells. Co-transfection of the puroΔtk transposon (FIG. 6, left) in the identified transposon systems with transposase expression constructs (FIG. 6, right);

FIGS. 7A-7I are data showing the presence of multicopy transgenic events in pig cells. 7A) generic transposon (pTP-PTK) used for colony formation. 7B) Graphic showing number of colonies formed using Tol2 transposon system; 7C) Graphic showing the number of colonies formed using the piggyback transposon system; 7D) Graphic showing the number of colonies formed using the Sleeping Beauty transposon system; 7E) Graphic showing the number of colonies formed using the Passport transposon system; 7F-7I Individual puromycin resistant PEGE colonies isolated and expanded for Southern analysis of the corresponding transposon systems in 7B-7E

FIGS. 8A-8C is micrograph of a Southern analysis of SM copy number in pigs; 8A) the Pkt2p-PuroΔtk transposon was used for selection of APOBEC3G and YFP-Cre cells by co-transpositional co-selection (CoCo). 8B) BamHI digestion of genomic DNA from APOBEC3G; 8C) BamHI digestion of genomic DNA from and YFP-Cre; founders would result in a 1.35-kb band (large black arrow) in animals harboring a concatemer insertion while transposase-mediated events are evident as slower-migrating fragments.

FIGS. 9A-9C Transgene copy number distribution in pigs and donor cells using co-transpositional co-selection (CoCo). 9A) 16 out of 27 (59%) pigs have at least one GOI (gene of interest) insertion, and the average insertion rate is 1.4 GOI insertions per founder; 9B) All founders carry at least one SM, as transfected cells were selected for antibiotic resistance prior to cloning. 9C) The sum of GOI and SM inserts in pigs should follow a Poisson distribution with a mean insertion number equal to the sum of the GOI and SM means (1.4+0.25=1.65), illustrated by the purple (upper) line.

FIGS. 10A-10C transposon coselection for indel enrichment. 10A) the Experimental timeline. 10B) Fibroblasts were transfected using Mirus LT1 reagent and Surveyor assay was performed on day 14 populations. 10C) Fibroblasts were transfected by nucleofections and the percent NHEJ was measured at day 3 and in day 14 nonelected (NS) and selected (S) populations. p FIGS. 11A-11B SM expression in APOBEC3G and YFP-Cre founders; 11A) SM expression (PuroΔtk) in tails of APOBEC3G founders. 11B) expression (PuroΔtk) in tails of YFP-Cre founders.

FIG. 12 provides some economic variables in transpositional transgenesis of pigs;

FIGS. 13A-C a cost analysis of transgenic pig production by multi-loci Tnt. 13A (right panel) The number of transgenes per founder (N) can be controlled by manipulating the transposon system, E—properly expressing transgene loci; F—number of founders. 13A (left panel) bionomial distribution of genotypes with the identified “N”. 13B) The total number of litters (Lₜₒₜₐₗ) required to isolate D=3 identical, transgene loci for each expression threshold can be found by multiplying the number of founders required by litters per founder. 13C) An economic model based on current costs for each component of transgenic line generation developed using the identified parameters.

FIG. 14 graphic showing the result on feral pig populations when the number of males expressing 2 copies of SRY is kept high, providing an estimate of the transposon resident sry for creating daughterless Boars for feral pig elimination.

### DETAILED DESCRIPTION OF THE EXEMPLARY EMBODIMENTS

The present invention provides methods to generate a livestock animal that is modified genetically to provide a sterile phenotype. Further, the method provides that the sterile livestock animal can be phenotypically either male or female. Such animals are beneficial in providing a genetically modified animal that is phenotypically male but sterile, such as a steer (cattle), gelding (horse), goat, sheep, pig or the like. Further the invention provides for producing phenotypically female animals that are also sterile. Sterile animals are useful not just for agricultural purposes but also to release into the wild for population control.

Unless defined otherwise, all technical and scientific terms used herein have the same meanings as commonly understood by one of ordinary skill in the art to which this invention belongs. All publications and patents specifically mentioned herein are incorporated by reference for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

“Additive Genetic Effects” as used herein means average individual gene effects that can be transmitted from parent to progeny.

“Allele” as used herein refers to an alternate form of a gene. It also can be thought of as variations of DNA sequence. For instance if an animal has the genotype for a specific gene of Bb, then both B and b are alleles.

“DNA Marker” refers to a specific DNA variation that can be tested for association with a physical characteristic.

“Genotype” refers to the genetic makeup of an animal.

“Genotyping (DNA marker testing)” refers to the process by which an animal is tested to determine the particular alleles it is carrying for a specific genetic test.

“Simple Traits” refers to traits such as coat color and horned status and some diseases that are carried by a single gene.

“Complex Traits” refers to traits such as reproduction, growth and carcass that are controlled by numerous genes.

“Complex allele”—coding region that has more than one mutation within it. This makes it more difficult to determine the effect of a given mutation because researchers cannot be sure which mutation within the allele is causing the effect.

“Copy number variation” (CNVs) a form of structural variation—are alterations of the DNA of a genome that results in the cell having an abnormal or, for certain genes, a normal variation in the number of copies of one or more sections of the DNA. CNVs correspond to relatively large regions of the genome that have been deleted (fewer than the normal number) or duplicated (more than the normal number) on certain chromosomes. For example, the chromosome that normally has sections in order as A-B-C-D might instead have sections A-B-C-C-D (a duplication of “C”) or A-B-D (a deletion of “C”).

“CoCo” as used herein refers to co-transpositional co-selection, or transposon co-selection. It is the act of using an unlinked selection marker (SM) transposon to select for the presence of a second, gene-of-interest transposon (GOI). This works since cells made transgenic with transposons typically have multiple copies of a transposon. Hence if transposon transgenesis starts with a 5:1 ratio of GOI to SM, it is very likely that any SM positive cell will also have the GOI.

“Repetitive element” patterns of nucleic acids (DNA or RNA) that occur in multiple copies throughout the genome. Repetitive DNA was first detected because of its rapid reassociation kinetics.

“Quantitative variation” variation measured on a continuum (e.g. height in human beings) rather than in discrete units or categories. See continuous variation. The existence of a range of phenotypes for a specific character, differing by degree rather than by distinct qualitative differences.

“Homozygous” refers to having two copies of the same allele for a single gene such as BB.

“Heterozygous” refers to having different copies of alleles for a single gene such as Bb.”

“Locus” (plural “loci”) refers to the specific locations of a maker or a gene.

“Centimorgan (Cm)” a unit of recombinant frequency for measuring genetic linkage.

“Marker Assisted Selection (MAS)” refers to the process by which DNA marker information is used to assist in making management decisions.

“Marker Panel” a combination of two or more DNA markers that are associated with a particular trait.

Jackpot” as used herein refers to multi-inserting or multi-edits in one culture at an early stage whereby the mutations or edits are cause by transposons or nucleases. It does not necessitate a mutation to be a jackpot, the jackpot may be due to a particular condition of the cell when treated; i.e. cell cycle stage specific or activation or suppression of some unidentified pathways.

“Non-additive Genetic Effects” refers to effects such as dominance and epistasis. Codominance is the interaction of alleles at the same locus while epistasis is the interaction of alleles at different loci.

As used herein there term “allosome” refers to a sex chromosome. In mammals, the sex chromosomes are the X chromosome and the Y chromosome.

As used herein the term “autosome” refers to any chromosome that is not a sex chromosome.

As used herein the term “allogeneic” refers to being genetically different but belonging to or obtained from the same species. As used herein, the term “xenogeneic” refers to being derived from a different species.

“Nucleotide” refers to a structural component of DNA that includes one of the four base chemicals: adenine (A), thymine (T), guanine (G), and cytosine (C).

“Phenotype” refers to the outward appearance of an animal that can be measured. Phenotypes are influenced by the genetic makeup of an animal and the environment.

“Single Nucleotide Polymorphism (SNP)” is a single nucleotide change in a DNA sequence.

“Haplotype” or “haploid genotype” refers to a combination of alleles, loci or DNA polymorphisms that are linked so as to cosegregate in a significant proportion of gametes during meiosis. The alleles of a haplotype may be in linkage disequilibrium (LD).

As used herein the term “ortholog” refers to a similar gene from a different species. Thus the term “ortholog gene” or “ortholog allele” refers to a gene or allele that has the same function in different species. For example a rat may be modified to express a sry gene from another species such as, for example, a goat. In contrast, the term “paralog” refers to the same gene or allele from the same species. For example the rat may be genomically modified to express a “sry allele” similar to sry alleles expressed by native members of its species.

“Linkage disequilibrium (LD)” is the non-random association of alleles at different loci i.e. the presence of statistical associations between alleles at different loci that are different from what would be expected if alleles were independently, randomly sampled based on their individual allele frequencies. If there is no linkage disequilibrium between alleles at different loci they are said to be in linkage equilibrium.

As used herein, the word “transposon” or “transposable element (TE)” refers to a DNA sequence that can change its position within a genome.

As used herein, “transposase” refers to an enzyme that binds to the end of a transposon and catalyzes the movement of the transposon to another part of the genome by a cut and paste mechanism or a replicative transposition mechanism.

The term “restriction fragment length polymorphism” or “RFLP” refers to any one of different DNA fragment lengths produced by restriction digestion of genomic DNA or cDNA with one or more endonuclease enzymes, wherein the fragment length vanes between individuals in a population.

“Introgression” also known as “introgressive hybridization”, is the movement of a gene (gene flow) from one species into the gene pool of another by the repeated backcrossing of an interspecific hybrid with one of its parent species. Purposeful introgression is a long-term process; it may take many hybrid generations before the backcrossing occurs.

As used herein the term “gene editing” or genome editing refers to a type of genetic engineering in which DNA is inserted, replaced, or removed from a genome using artificially engineered nucleases, or “molecular scissors”. The nucleases create specific double-strand breaks (DSBs) at desired locations in the genome, and harness the cell's endogenous mechanisms to repair the induced break by natural processes of homologous recombination (HR) and nonhomologous end-joining (NHEJ). There are currently four families of engineered nucleases being used: Zinc finger nucleases (ZFNs), Transcription Activator-Like Effector Nucleases (TALENs), the CRISPR/Cas system, and engineered meganuclease re-engineered homing endonucleases.

“Nonmeiotic introgression” genetic introgression via introduction of a gene or allele in a diploid (non-gametic) cell. Non-meiotic introgression does not rely on sexual reproduction and does not require backcrossing and, significantly, is carried out in a single generation.

“Transcription activator-like effector nucleases (TALENs)” are artificial restriction enzymes generated by fusing a TAL effector DNA-binding domain to a DNA cleavage domain.

Zinc Finger Nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes.

CRISPR/CAS (Clustered Regularly Interspaced Short Palindromic Repeats/CRISPR associated nucleases) system has been used for gene editing and gene regulation in species throughout the tree of life. By delivering the Cas9 protein and appropriate guide RNAs into a cell, the organism's genome can be cut at any desired location.

As used herein the term “ssODN” refers to a single-stranded donor oligonucleotide or a single stranded DNA oligonucleotides are used to direct gene repair after a double strand nick has been induced in a gene using both CRISPR/Cas9 and TALENs.

“Meganuclease” as used herein are another technology useful for gene editing and are endodeoxyribonucleases characterized by a large recognition site (double-stranded DNA sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes.

“Indel” as used herein is shorthand for “insertion” or “deletion” referring to a modification of the DNA in an organism.

“Genetic marker” as used herein refers to a gene/allele or known DNA sequence with a known location on a chromosome. The markers may be any genetic marker e.g., one or more alleles, haplotypes, haplogroups, loci, quantitative trait loci, or DNA polymorphisms restriction fragment length polymorphisms(RFLPs), amplified fragment length polymorphisms (AFLPs), single nuclear polymorphisms (SNPs), indels, short tandem repeats (STRs), microsatellites and minisatellites. Conveniently, the markers are SNPs or STRs such as microsatellites, and more preferably SNPs. Preferably, the markers within each chromosome segment are in linkage disequilibrium.

As used herein the term “host animal” means an animal which has a native genetic complement of a recognized species or breed of animal.

As used herein, the terms “native allele”, “native haplotype” or “native genome” means the natural DNA of a particular species or breed of animal that is chosen to be the recipient of a gene or allele that is not present in the host animal e.g., a “foreign” allele.

As used herein, the term “introduced allele” or “introduced copy” refers to a foreign allele that is introduced into an animal's genome using non-meiotic introgression. The introduced allele may be from the same breed and the same species as the host. The introduced allele may be from a different species from the host. However, generally the introduced allele is transferred into the genome at a different site compared to the native allele. For example, a bull (Bos taurus) having an XY genotype will have a native sry allele located on the Y chromosome at, for example chromosome Y (13), AC_000170.1. Similarly, a boar (Sus scrofa) will have a sry allele on the Y chromosome, NC_010462.2). “Introduced” sry alleles whether from the same species as the host or from different species will be found in different locations than those mapped for native sry. For example, an introduced sry allele may be found on any chromosome other than the Y chromosome. Further a sry allele may also be found on the Y chromosome but the introduced sry allele will not be found at AC_000170.1. Similarly a pig having an introduced sry allele, may have the inserted sry allele anywhere in its genome except at NC_010462.2, which is identified as the native sry allele.

As used herein the term “genetic modification” refers to is the direct manipulation of an organism genome using biotechnology.

As used herein the term “wildtype” (WT) is used to refer to the phenotype of the typical form of a species as it occurs in nature. As used herein, the term applies to a genomic complement as it existed before a genomic modification event.

As used herein, the term “parental generation” (Po) refers to an organism before a genetic modification; in many cases the Po generation will have a WT genome. As used herein, the term “F1” refers to a first filial generation, e.g., the first generation encompassing a genetic modification event. The term “F2” refers to a second filial generation produced by the F1 generation. “F3” refers to a third filial generation produced by the F2 generation et cetera.

As used herein “Therian” refers to a subclass of mammals that give birth to live young without using a shelled egg including marsupials and mammals. Therians are the only animals whose gender is determined by the XY/XX genetic chromosomal system. In therians, individuals having two X chromosomes (XX) are female and individuals having one X and one Y chromosome are considered males.

As used herein, the term “zygote” refers to a fertilized egg.

As used herein the term “target locus” means a specific location of a known allele on a chromosome.

As used herein, the term “quantitative trait locus (QTL)” is a section of DNA (the locus) that correlates with variation in a phenotype (the quantitative trait).

As used herein the term “cloning” means production of genetically identical organisms asexually.

“Somatic cell nuclear transfer” (“SCNT”) is one strategy for cloning a viable embryo from a body cell and an egg cell. The technique consists of taking an enucleated oocyte (egg cell) and implanting a donor nucleus from a somatic (body) cell.

“Zygote microinjection” as used herein refers to a method to prepare transgenic animals. In “pronuclear microinjection”, foreign DNA is physically injected into the pronuclei of fertilized eggs using pulled glass needles. Another method is “cytoplasmic microinjection”. Both injection locations allow for injection of a variety of things, transposons, HR templates, TALENs, ZFN CRISPR in DNA, mRNA or protein forms. Using these techniques, microinjection, can now be specifically targeted or enzymatic via transposition. As with pro-nuclear microinjection using transposons, co-injection into the cytoplasm of a plasmid encoding a hyperactive transposase, together with a second plasmid carrying a transgene flanked by binding sites for the transposase into the cytoplasm of a zygote is effective. The transposase mediates excision of the transgene cassette from the plasmid vector and its permanent insertion into the genome to produce stable transgenic animals.

“Genotyping” or “genetic testing” generally refers to detecting one or more markers of interest e.g., SNPs in a sample from an individual being tested, and analyzing the results obtained to determine the haplotype of the subject. As will be apparent from the disclosure herein, it is particularly preferred to detect the one or more markers of interest using a high-throughput system comprising a solid support consisting essentially of or having nucleic acids of different sequence bound directly or indirectly thereto, wherein each nucleic acid of different sequence comprises a polymorphic genetic marker derived from an ancestor or founder that is representative of the current population and, more preferably wherein said high-throughput system comprises sufficient markers to be representative of the genome of the current population. Preferred samples for genotyping comprise nucleic acid, e.g., RNA or genomic DNA and preferably genomic DNA.

Genetic testing of animals can be performed using a hair follicle, for example, isolated from the tail of an animal to be tested. Other examples of readily accessible samples include, for example, skin or a bodily fluid or an extract thereof or a fraction thereof. For example, a readily accessible bodily fluid includes, for example, whole blood, saliva, semen or urine. Exemplary whole blood fractions are selected from the group consisting of buffy-coat fraction, Fraction II+III obtainable by ethanol fractionation of Cohn (E. J. Cohn et al., J. Am. Chem. Soc., 68, 459 (1946), Fraction II obtainable by ethanol fractionation of Cohn (E. J. Cohn et al., J. Am. Chem. Soc., 68, 459 (1946), albumin fraction, an immunoglobulin-containing fraction and mixtures thereof, Preferably, a sample from an animal has been isolated or derived previously from an animal subject by, for example, surgery, or using a syringe or swab.

A sample can comprise a cell or cell extract or mixture thereof derived from a tissue or organ such as described herein above. Nucleic acid preparation derived from organs, tissues or cells are also particularly useful. The sample can be prepared on a solid matrix for histological analyses, or alternatively, in a suitable solution such as, for example, an extraction buffer or suspension buffer, and the present invention clearly extends to the testing of biological solutions thus prepared. However, in a preferred embodiment, the high-throughput system of the present invention is employed using samples in solution.

In other exemplary embodiments according to the invention, an animal thought to have been produced by genetic manipulation can be tested to determine whether a trait exhibited by that animal is due to sexual breeding or whether the trait is present due to genetic modification and the animal subsequently cloned, such as by SCNT or pronuclear microinjection.

Accordingly, the skilled artisan can design probes and/or primers to determine the origin of a phenotypic or genotypic trait. The skilled artisan is aware that a suitable probe or primer i.e., one capable of specifically detecting a marker or foreign allele at a target locus, will specifically hybridize to a region of the genome in genomic DNA from the individual being tested that comprises the marker or allele. As used herein “selectively hybridizes” means that the polynucleotide used as a probe is used under conditions where a target polynucleotide is found to hybridize to the probe at a level significantly above background. The background hybridization may occur because of other polynucleotides present, for example, in genomic DNA being screened. In this event, background implies a level of signal generated by interaction between the probe and non-specific DNA which is less than 10 fold, preferably less than 100 fold as intense as the specific interaction observed with the target DNA. The intensity of interaction are measured, for example, by radiolabeling the probe, e.g. with ³²P.

As will be known to the skilled artisan a probe or primer comprises nucleic acid and may consist of synthetic oligonucleotides up to about 100-300 nucleotides in length and more preferably of about 50-100 nucleotides in length and still more preferably at least about 8-100 or 8-50 nucleotides in length. For example, locked nucleic acid (LNA) or protein-nucleic acid (PNA) probes or molecular beacons for the detection of one or more SNPs are generally at least about 8 to 12 nucleotides in length. Longer nucleic acid fragments up to several kilo bases in length can also be used, e.g., derived from genomic DNA that has been sheared or digested with one or more restriction endonucleases. Alternatively, probes/primers can comprise RNA.

Preferred probes or primers for use in the present disclosure will be compatible with the high-throughput system described herein. Exemplary probes and primers will comprise locked nucleic acid (LNA) or protein-nucleic acid (PNA) probes or molecular beacons, preferably bound to a solid phase. For example, LNA or PNA probes bound to a solid support are used, wherein the probes each comprise an SNP and sufficient probes are bound to the solid support to span the genome of the species to which an individual being tested belongs.

The number of probes or primers will vary depending upon the number of loci or QTLs being screened and, in the case of genome-wide screens, the size of the genome being screened. The determination of such parameters is readily determined by a skilled artisan without undue experimentation.

Specificity of probes or primers can also depend upon the format of hybridization or amplification reaction employed for genotyping.

The sequence(s) of any particular probe(s) or primer(s) used in the method of the present invention will depend upon the locus or QTL or combination thereof being screened. In this respect, the present invention can be generally applied to the genotyping of any locus or QTL or to the simultaneous or sequential genotyping of any number of QTLs or loci including genome-wide genotyping. This generality is not to be taken away or read down to a specific locus or QTL or combination thereof. The determination of probe/primer sequences is readily determined by a skilled artisan without undue experimentation

Standard methods are employed for designing probes and/or primers e.g., as described by Dveksler (Eds) (In: PCR Primer: A Laboratory Manual, Cold Spring Harbour Laboratories, NY, 1995). Software packages are also publicly available for designing optimal probes and/or primers for a variety of assays, e.g., Primer 3 available from the Center for Genome Research, Cambridge, Mass., USA. Probes and/or primers are preferably assessed to determine those that do not form hairpins, self-prime, or form primer dimers (e.g. with another probe or primer used in a detection assay). Furthermore, a probe or primer (or the sequence thereof) is preferably assessed to determine the temperature at which it denatures from a target nucleic acid (i.e. the melting temperature of the probe or primer, or Tm). Methods of determining Tm are known in the art and described, for example, in Santa Lucia, Proc. Natl. Acad. Sci. USA, 95: 1460-1465, 1995 or Bresslauer et al., Proc. Natl. Acad. Sci. USA, 83: 3746-3750, 1986.

For LNA or PNA probes or molecular beacons, it is particularly preferred for the probe or molecular beacon to be at least about 8 to 12 nucleotides in length and more preferably, for the SNP to be positioned at approximately the center of the probe, thereby facilitating selective hybridization and accurate detection.

For detecting one or more SNPs using an allele-specific PCR assay or a ligase chain reaction assay, the probe/primer is generally designed such that the 3′ terminal nucleotide hybridizes to the site of the SNP. The 3′ terminal nucleotide may be complementary to any of the nucleotides known to be present at the site of the SNP. When complementary nucleotides occur in both the probe/primer and at the site of the polymorphism, the 3′ end of the probe or primer hybridizes completely to the marker of interest and facilitates, for example, PCR amplification or ligation to another nucleic acid. Accordingly, a probe or primer that completely hybridizes to the target nucleic acid produces a positive result in an assay.

For primer extension reactions, the probe/primer is generally designed such that it specifically hybridizes to a region adjacent to a specific nucleotide of interest, e.g., an SNP. While the specific hybridization of a probe or primer may be estimated by determining the degree of homology of the probe or primer to any nucleic acid using software, such as, for example, BLAST, the specificity of a probe or primer is generally determined empirically using methods known in the art.

Methods of producing/synthesizing probes and/or primers useful in the present invention are known in the art. For example, oligonucleotide synthesis is described, in Gait (Ed) (In: Oligonucleotide Synthesis: A Practical Approach, IRL Press, Oxford, 1984); LNA synthesis is described, for example, in Nielsen et al, J. Chem. Soc. Perkin Trans., 1: 3423, 1997; Singh and Wengel, Chem. Commun. 1247, 1998; and PNA synthesis is described, for example, in Egholm et al., Am. Chem. Soc., 114: 1895, 1992; Egholm et al., Nature, 365: 566, 1993; and Orum et al., Nucl. Acids Res., 21: 5332, 1993.

A variety of nucleic acids may be introduced into the artiodactyl or other cells, for knockout purposes, or to obtain expression of a gene for other purposes. Nucleic acid constructs that can be used to produce transgenic animals include a target nucleic acid sequence. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller (1997) Antisense Nucleic Acid Drug Dev. 7(3):187; and Hyrup et al. (1996) Bioorgan. Med. Chem. 4:5. In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, and promoters responsive or unresponsive to a particular stimulus e.g., inducible promoters. As used herein, a constitutive promoter is a promoter that is active in all circumstances in the cell. Tissue specific promoters can result in preferential expression of a nucleic acid transcript in beta cells and include, for example, the human insulin promoter. Other tissue specific promoters can result in preferential expression in, for example, hepatocytes or heart tissue and can include the albumin or alpha-myosin heavy chain promoters, respectively. In other embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al. (2001) Hum. Gene Ther. 12:563; and Kiwaki et al. (1996) Hum. Gene Ther. 7:821.

An example of an inducible promoter is the tetracycline (tet)-on promoter system, which can be used to regulate transcription of the nucleic acid. In this system, a mutated Tet repressor (TetR) is fused to the activation domain of herpes simplex virus VP16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent. Other inducible promoters include the Hsp70.3, LAC, TRE.

Constitutive promoters include CMV, CaMV 35s, SV40, CMV, UBC, EF1A, PGK and CAGG.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. (1992) 89:6861, for a review of Cre/lox technology, and Brand and Dymecki, Dev. Cell (2004) 6:7. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in FO animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the target nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

In other embodiments, the target nucleic acid sequence induces RNA interference against a target nucleic acid such that expression of the target nucleic acid is reduced. For example the target nucleic acid sequence can induce RNA interference against a nucleic acid encoding a cystic fibrosis transmembrane conductance regulatory (CFTR) polypeptide. For example, double-stranded small interfering RNA (siRNA) or small hairpin RNA (shRNA) homologous to a CFTR DNA can be used to reduce expression of that DNA. Constructs for siRNA can be produced as described, for example, in Fire et al. (1998) Nature 391:806; Romano and Masino (1992) Mol. Microbiol. 6:3343; Cogoni et al. (1996) EMBO J. 15:3153; Cogoni and Masino (1999) Nature 399:166; Misquitta and Paterson (1999) Proc. Natl. Acad. Sci. USA 96:1451; and Kennerdell and Carthew (1998) Cell 95:1017. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

Nucleic acid constructs can be methylated using an SssI CpG methylase (New England Biolabs, Ipswich, Mass.). In general, the nucleic acid construct can be incubated with S-adenosylmethionine and SssI CpG-methylase in buffer at 37° C. Hypermethylation can be confirmed by incubating the construct with one unit of HinP1I endonuclease for 1 hour at 37° C. and assaying by agarose gel electrophoresis.

Nucleic acid constructs can be introduced into embryonic, fetal, or adult artiodactyl cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

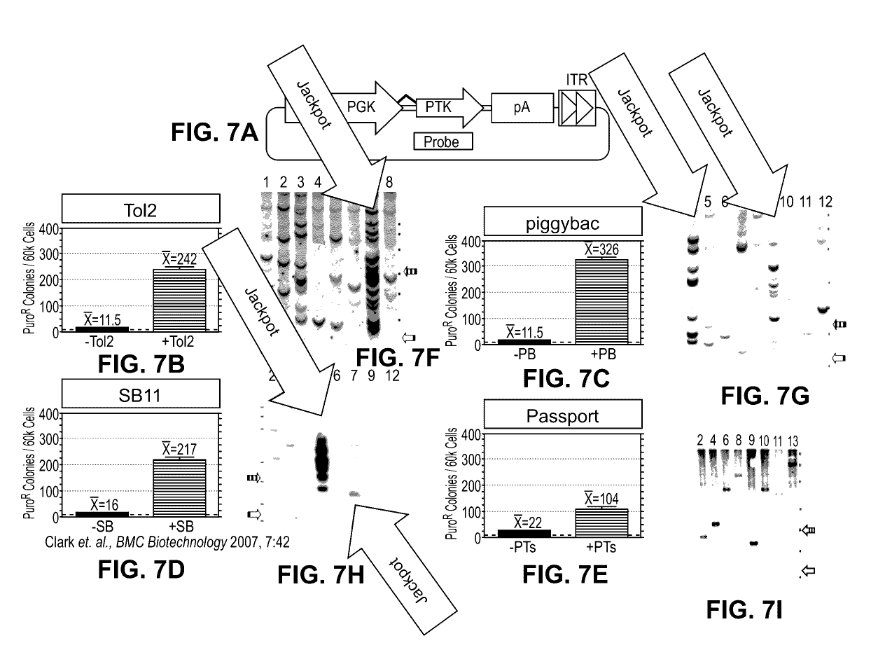

In transposon systems, the transcriptional unit of a nucleic acid construct, i.e., the regulatory region operably linked to a target nucleic acid sequence, is flanked by an inverted repeat of a transposon. In general, transposon systems consist of two components: (1) the transposon vector that contains a transgenic expression cassette flanked by inverted terminal repeats (ITRs); and (2) a source for the transposase enzyme that is generally provided by either a second gene on the same (cis) or separate (trans) vector or mRNA. Several transposon systems, including, for example, Sleeping Beauty (see, U.S. Pat. No. 6,613,752 and U.S. Publication No. 2005/0003542); Frog Prince (Miskey et al. (2003) Nucleic Acids Res. 31:6873); Tol2 (Kawakami (2007) Genome Biology 8(Supp1.1):57; Minos (Pavlopoulos et al. (2007) Genome Biology 8(Supp1.1):S2); Hsmar1 (Miskey et al. (2007)) Mol Cell Biol. 27:4589); Piggybac (Clark et al., BMC Biotechnol. 2007 Jul 17;7:42) and Passport have been developed to introduce nucleic acids into cells, including mice, human, and pig cells. Further, these systems have been shown to be able to insert multiple copies of transgenes into a genome randomly, a phenomenon referred to as a “Jackpot”, FIG. 6. A transposase can be delivered as a protein, encoded on the same nucleic acid construct as the target nucleic acid, can be introduced on a separate nucleic acid construct, or provided as an mRNA (e.g., an in vitro-transcribed and capped mRNA). Further the use of transposon systems have the added benefit that cells and animal cloned therefrom will not have the introduction of selectable markers, such as, antibiotic resistance genes, fluorophores and the like, inserted into the genome for selection (Carlson et al., Trans Res 2011 Oct;20(5):1125-37). In addition, the use of transposon systems limits the occurrence of concatemerization of transgenes that occurs using traditional methods of transgene insertion.

Numerous methods are known in the art for detecting the occurrence of a particular marker in a sample. In one embodiment, a marker is detected using a probe or primer that selectively hybridizes to said marker in a sample from an individual under moderate stringency, and preferably, high stringency conditions. If the probe or primer is detectably labelled with a suitable reporter molecule, e.g., a chemiluminescent label, fluorescent label, radiolabel, enzyme, hapten, or unique oligonucleotide sequence etc., then the hybridization may be detected directly by determining binding of reporter molecule. Alternatively, hybridized probe or primer may be detected by performing an amplification reaction such as polymerase chain reaction (PCR) or similar format, and detecting the amplified nucleic acid. Preferably, the probe or primer is bound to solid support e.g., in the high-throughput system of the present invention.

For the purposes of defining the level of stringency to be used in the hybridization, a low stringency is defined herein as hybridization and/or a wash step(s) carried out in 2-6xSSC buffer, 0.1% (w/v) SDS at 28° C., or equivalent conditions. A moderate stringency is defined herein as hybridization and/or a wash step(s) carried out in 0.2-2x SSC buffer, 0.1% (w/v) SDS at a temperature in the range 45° C. to 65° C., or equivalent conditions. A high stringency is defined herein as hybridization and/or a wash step(s) carried out in 0.1x SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, or the type of hybridization probe used.

Progressively higher stringency conditions can also be employed wherein the stringency is increased stepwise from lower to higher stringency conditions. Exemplary progressive stringency conditions are as follows: 2xSSC/0.1% SDS at about room temperature (hybridization conditions); 0.2xSSC/0.1% SDS at about room temperature (low stringency conditions); 0.2xSSC/0.1% SDS at about 42° C., (moderate stringency conditions); and 0.1xSSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

For example, the modification of a sequence of a region (haplotype) of the genome or an expression product thereof, such as, for example, an insertion (e.g., introduction of a foreign allele at a target locus), a deletion, a transversion or a transition, is detected using a method, such as, polymerase chain reaction (PCR), strand displacement amplification, ligase chain reaction, cycling probe technology or a DNA microarray chip amongst others.

Methods of PCR are known in the art and described, for example, in Dieffenbach (ed) and Dveksler (ed) (In: PCR Primer: A Laboratory Manual, Cold Spring Harbour Laboratories, NY, 1995). Generally, for PCR two non-complementary nucleic acid primer molecules comprising at least about 15 nucleotides, more preferably at least 20 nucleotides in length are hybridized to different strands of a nucleic acid template molecule, and specific nucleic acid molecule copies of the template are amplified enzymatically. PCR products may be detected using electrophoresis and detection with a detectable marker that binds nucleic acids. Alternatively, one or more of the oligonucleotides is/are labeled with a detectable marker (e.g. a fluorophore) and the amplification product detected using, for example, a lightcycler (Perkin Elmer, Wellesley, Mass., USA). Clearly, the present invention also encompasses quantitative forms of PCR, such as, for example, Taqman assays.

Strand displacement amplification (SDA) utilizes oligonucleotides, a DNA polymerase and a restriction endonuclease to amplify a target sequence. The oligonucleotides are hybridized to a target nucleic acid and the polymerase used to produce a copy of this region. The duplexes of copied nucleic acid and target nucleic acid are then nicked with an endonuclease that specifically recognizes a sequence at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

Ligase chain reaction (described, for example, in EP 320,308 and U.S. Pat. No. 4,883,750) uses at least two oligonucleotides that bind to a target nucleic acid in such a way that they are adjacent. A ligase enzyme is then used to link the oligonucleotides. Using thermocycling the ligated oligonucleotides then become a target for further oligonucleotides. The ligated fragments are then detected, for example, using electrophoresis, or MALDI-TOF. Alternatively, or in addition, one or more of the probes is labeled with a detectable marker, thereby facilitating rapid detection.

Cycling Probe Technology uses chimeric synthetic probe that comprises DNA-RNA-DNA that is capable of hybridizing to a target sequence. Upon hybridization to a target sequence the RNA-DNA duplex formed is a target for RNase H thereby cleaving the probe. The cleaved probe is then detected using, for example, electrophoresis or MALDI-TOF.

Additional methods for detecting SNPs are known in the art, and reviewed, for example, in Landegren et al, Genome Research 8: 769-776, 1998)(hereby incorporated by reference in its entirety).

For example, an SNP that introduces or alters a sequence that is a recognition sequence for a restriction endonuclease is detected by digesting DNA with the endonuclease and detecting the fragment of interest using, for example, Southern blotting (described in Ausubel et al (In: Current Protocols in Molecular Biology. Wiley Interscience, ISBN 047 150338, 1987) (herein incorporated by reference in its entirety) and Sambrook et al (In: Molecular Cloning: Molecular Cloning: A Laboratory Manual, Cold Spring Harbor Laboratories, New York, Third Edition 2001) (herein incorporated by reference in its entirety). Alternatively, a nucleic acid amplification method described supra, is used to amplify the region surrounding the SNP. The amplification product is then incubated with the endonuclease and any resulting fragments detected, for example, by electrophoresis, MALDI-TOF or PCR.

The direct analysis of the sequence of polymorphisms of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988) (incorporated herein by reference in its entirety). For example, a region of genomic DNA comprising one or more markers is amplified using an amplification reaction, e.g., PCR, and following purification of the amplification product, the amplified nucleic acid is used in a sequencing reaction to determine the sequence of one or both alleles at the site of an SNP of interest.

Alternatively, one or more SNPs is/are detected using single stranded conformational polymorphism (SSCP). SSCP relies upon the formation of secondary structures in nucleic acids and the sequence dependent nature of these secondary structures. In one form of this analysis, an amplification method, such as, for example, a method described supra, is used to amplify a nucleic acid that comprises an SNP. The amplified nucleic acids are then denatured, cooled and analyzed using, for example, non-denaturing polyacrylamide gel electrophoresis, mass spectrometry, or liquid chromatography (e.g., HPLC or dHPLC). Regions that comprise different sequences form different secondary structures, and as a consequence migrate at different rates through, for example, a gel and/or a charged field. Clearly, a detectable marker may be incorporated into a probe/primer useful in SSCP analysis to facilitate rapid marker detection.

Alternatively, any nucleotide changes may be detected using, for example, mass spectrometry or capillary electrophoresis. For example, amplified products of a region of DNA comprising an SNP from a test sample are mixed with amplified products from an individual having a known genotype at the site of the SNP. The products are denatured and allowed to re-anneal. Those samples that comprise a different nucleotide at the position of the SNP will not completely anneal to a nucleic acid molecule from the control sample thereby changing the charge and/or conformation of the nucleic acid, when compared to a completely annealed nucleic acid. Such incorrect base pairing is detectable using, for example, mass spectrometry.

Allele-specific PCR (as described, for example, In Liu et al, Genome Research, 7: 389-398, 1997) (herein incorporated by reference in its entirety) is also useful for determining the presence of one or other allele of an SNP. An oligonucleotide is designed, in which the most 3′ base of the oligonucleotide hybridizes to a specific form of an SNP of interest (i.e., allele). During a PCR reaction, the 3′ end of the oligonucleotide does not hybridize to a target sequence that does not comprise the particular form of the SNP detected. Accordingly, little or no PCR product is produced, indicating that a base other than that present in the oligonucleotide is present at the site of SNP in the sample. PCR products are then detected using, for example, gel or capillary electrophoresis or mass spectrometry.

Primer extension methods (described, for example, in Dieffenbach (ed) and Dveksler (ed) (In: PCR Primer: A Laboratory Manual, Cold Spring Harbour Laboratories, NY, 1995)) are also useful for the detection of an SNP. An oligonucleotide is used that hybridizes to the region of a nucleic acid adjacent to the SNP. This oligonucleotide is used in a primer extension protocol with a polymerase and a free nucleotide diphosphate that corresponds to either or any of the possible bases that occur at the site of the SNP. Preferably, the nucleotide-diphosphate is labeled with a detectable marker (e.g. a fluorophore). Following primer extension, unbound labeled nucleotide diphosphates are removed, e.g. using size exclusion chromatography or electrophoresis, or hydrolyzed, using for example, alkaline phosphatase, and the incorporation of the labeled nucleotide into the oligonucleotide is detected, indicating the base that is present at the site of the SNP. Alternatively, or in addition, as exemplified herein primer extension products are detected using mass spectrometry (e.g., MALDI-TOF).

### Homology Directed Repair (HDR)

Homology directed repair (HDR) is a mechanism in cells to repair nicked DNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Gal4 motifs. Introgression of an allele refers to a process of copying an exogenous allele over an endogenous allele with a template-guided process. The endogenous allele might actually be excised and replaced by an exogenous nucleic acid allele in some situations but present theory is that the process is a copying mechanism. Since alleles are gene pairs, there is significant homology between them. The allele might be a gene that encodes a protein, or it could have other functions such as encoding a bioactive RNA chain or providing a site for receiving a regulatory protein or RNA.

The HDR template is a nucleic acid that comprises the allele that is being introgressed. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences that provide homology to DNA adjacent to the endogenous allele or the DNA that is to be replaced. The template may also comprise a sequence that is bound to a targeted nuclease system, and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site.

### Targeted Endonuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is an RGEN. tracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### TALENs

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The cipher for TALs has been reported (PCT Publication WO 2011/072246) wherein each DNA binding repeat is responsible for recognizing one base pair in the target DNA sequence. The residues may be assembled to target a DNA sequence. In brief, a target site for binding of a

TALEN is determined and a fusion molecule comprising a nuclease and a series of repeat variable diresidue “RVDs”, the 12^{th} and 13^{th} amino acids that are highly variable in an otherwise highly conserved 33-34 amino acid binding domain of the TALENs. These two locations (12 and 13) are highly variable and show a strong correlation with specific nucleotide recognition. Upon binding, the nuclease cleaves the DNA so that cellular repair machinery can operate to make a genetic modification at the cut ends. The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvCl, EcoRI, BglII, and AlwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a targeted endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu L I-Cre I, I-Csm I, PI-See L PI-Tti L PI-Mtu I, I-Ceu I, I-See IL 1-See III, HO, PI-Civ I, PI-Ctr L PI-Aae I, PI-Bsu I, PI-Dha I, PI-Dra L PI-Mav L PI-Meh I, PI-Mfu L PI-Mfl I, PI-Mga L PI-Mgo I, PI-Min L PI-Mka L PI-Mle I, PI-Mma I, PI-30 Msh L PI-Msm I, PI-Mth I, PI-Mtu I, PI-Mxe I, PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-MsoI.

A genetic modification made by TALENs or other tools may be, for example, chosen from the list consisting of an insertion, a deletion, insertion of an exogenous nucleic acid fragment, and a substitution. The term insertion is used broadly to mean either literal insertion into the chromosome or use of the exogenous sequence as a template for repair. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting non-human vertebrates, non-human primates, cattle, horse, swine, sheep, chicken, avian, rabbit, goats, dog, cat, laboratory animal, and fish.

Some embodiments involve a composition or a method of making a genetically modified livestock and/or artiodactyl comprising introducing a TALEN-pair into livestock and/or an artiodactyl cell or embryo that makes a genetic modification to DNA of the cell or embryo at a site that is specifically bound by the TALEN-pair, and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods. The phrase “a genetic modification to DNA of the cell at a site that is specifically bound by the TALEN”, or the like, means that the genetic modification is made at the site cut by the nuclease on the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic modification comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to alter the genomes of higher organisms. ZFNs may be used in method of inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific FokI cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM). Materials and methods for using zinc fingers and zinc finger nucleases for making genetically modified animals are disclosed in, e.g., U.S. Pat. No. 8,106,255; U.S. 2012/0192298; U.S. 2011/0023159; and U.S. 2011/0281306.

### Vectors and Nucleic Acids

A variety of nucleic acids may be introduced into cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

In general, type of promoter can be operably linked to a target nucleic acid sequence. Examples of promoters include, without limitation, tissue-specific promoters, constitutive promoters, inducible promoters, and promoters responsive or unresponsive to a particular stimulus. In some embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3 -phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al., Hum. Gene Ther. 12:563, 2001; and Kiwaki et al., Hum. Gene Ther. 7:821, 1996.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable expressed markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban et al., Proc. Natl. Acad. Sci., 89:6861, 1992, for a review of Cre/lox technology, and Brand and Dymecki, Dev. Cell, 6:7, 2004. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be introduced into embryonic, fetal, or adult artiodactyl/livestock cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. 2005/0003542); Frog Prince (Miskey et al., Nucleic Acids Res., 31:6873, 2003); Tol2 (Kawakami, Genome Biology, 8(Supp1.1):57, 2007); Minos (Pavlopoulos et al., Genome Biology, 8(Supp1.1):52, 2007); Hsmar1 (Miskey et al., Mol Cell Biol., 27:4589, 2007); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically modified organism or genetically engineered organism whose genetic material has been altered using genetic engineering techniques. A knockout artiodactyl is thus transgenic regardless of whether or not exogenous genes or nucleic acids are expressed in the animal or its progeny.

### Genetically Modified Animals

Animals may be modified using TALENs or other genetic engineering tools, including recombinase fusion proteins, or various vectors that are known. A genetic modification made by such tools may comprise disruption of a gene. The term disruption of a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying animals are further detailed in U.S. Pat. No. 8,518,701; U.S. 2010/0251395; and U.S. 2012/0222143 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation the target gene. The trans-acting gene may be on the same chromosome as the target gene, but the activity is via the intermediary protein or RNA that it encodes. Embodiments of trans-acting gene are, e.g., genes that encode targeting endonucleases. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knock-out animals and/or to introduce nucleic acid constructs into animals to produce founder animals and to make animal lines, in which the knockout or nucleic acid construct is integrated into the genome.

Such techniques include, without limitation, pronuclear microinjection (U.S. Pat. No. 4,873,191), retrovirus mediated gene transfer into germ lines (Van der Putten et al., Proc. Natl. Acad. Sci. USA, 82:6148-6152, 1985), gene targeting into embryonic stem cells (Thompson et al., Cell, 56:313-321, 1989), electroporation of embryos (Lo, Mol. Cell. Biol., 3:1803-1814, 1983), sperm-mediated gene transfer (Lavitrano et al., Proc. Natl. Acad. Sci. USA, 99:14230-14235, 2002; Lavitrano et al., Reprod. Fert. Develop., 18:19-23, 2006), and in vitro transformation of somatic cells, such as cumulus or mammary cells, or adult, fetal, or embryonic stem cells, followed by nuclear transplantation (Wilmut et al., Nature, 385:810-813, 1997; and Wakayama et al., Nature, 394:369-374, 1998). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for instance, may be used to make a mosaic animal if its cells are modified at the blastocyst state, or genomic modification can take place when a single-cell is modified. Animals that are modified so they do not sexually mature can be homozygous or heterozygous for the modification, depending on the specific approach that is used. If a particular gene is inactivated by a knock out modification, homozygosity would normally be required. If a particular gene is inactivated by an RNA interference or dominant negative strategy, then heterozygosity is often adequate.

In zygote microinjection, a nucleic acid construct is introduced into a fertilized egg or zygote; typically at the stage where pronuclei containing the genetic material from the sperm head and the egg are visible within the protoplasm or up to the two cell stage. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute. In cytoplasmic microinjection a variety of elements can be injected including transposons, HR templates, TALENs, ZFN, CRISPR in DNA, mRNA or protein forms. Using these techniques, specific nucleic acid sequences can be targeted or random enzymatic insertion via transposition can be achieved.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×10⁵ sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Nucleic acid constructs can be injected into one of the pronuclei in a variety of conformations including supercoiled, ssDNA dsDNA, linear and circular, as well as RNA and protein. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al., Science, 280:1256-1258, 1998; and U.S. 6,548,741. For pigs, recipient females can be checked for pregnancy approximately 20-21 days after transfer of the embryos.

Standard breeding techniques can be used to create animals that are homozygous for the exogenous nucleic acid from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animal have been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., Molecular Cloning, A Laboratory Manual, second edition, Cold Spring Harbor Press, Plainview; NY., 1989. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example

PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis, Genetic Engineering News, 12:1, 1992; Guatelli et al., Proc. Natl. Acad. Sci. USA, 87:1874, 1990; and Weiss, Science, 254:1292, 1991. At the blastocyst stage, embryos can be individually processed for analysis by PCR, Southern hybridization and splinkerette PCR (see, e.g., Dupuy et al., Proc Natl Acad Sci USA, 99:4495, 2002).

Expression of a nucleic acid sequence encoding a polypeptide in the tissues of transgenic pigs can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of disrupting a gene in a genetically modified animal comprises inducing RNA interference against a target gene and/or nucleic acid such that expression of the target gene and/or nucleic acid is reduced.

For example the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al., Nature, 391:806, 1998; Romano and Masino, Mol. Microbiol., 6:3343, 1992; Cogoni et al., EMBO J., 15:3153, 1996; Cogoni and Masino, Nature, 399:166, 1999; Misquitta and Paterson Proc. Natl. Acad. Sci. USA, 96:1451, 1999; and Kennerdell and Carthew, Cell, 95:1017, 1998. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically modified animal such as a livestock animal that express an RNAi directed against a gene, e.g., a gene selective for a developmental stage. The RNAi may be, for instance, selected from the group consisting of siRNA, shRNA, dsRNA, RISC and miRNA.

### Inducible Systems

An inducible system may be used to control expression of a gene. Various inducible systems are known that allow spatiotemporal control of expression of a gene. Several have been proven to be functional in vivo in transgenic animals. The term inducible system includes traditional promoters and inducible gene expression elements.

An example of an inducible system is the tetracycline (tet)-on promoter system, which can be used to regulate transcription of the nucleic acid. In this system, a mutated Tet repressor (TetR) is fused to the activation domain of herpes simplex virus VP16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two strains of mice provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a floxed stop codon is inserted between the promoter sequence and the DNA of interest. Genetically modified animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically modified animal such as a livestock animal that comprise a gene under control of an inducible system. The genetic modification of an animal may be genomic or mosaic. The inducible system may be, for instance, selected from the group consisting of Tet-On, Tet-Off, Cre-lox, and Hifl1alpha. An embodiment is a gene set forth herein.

### Dominant Negatives

Genes may thus be disrupted not only by removal or RNAi suppression but also by creation/expression of a dominant negative variant of a protein which has inhibitory effects on the normal function of that gene product. The expression of a dominant negative (DN) gene can result in an altered phenotype, exerted by a) a titration effect; the DN PASSIVELY competes with an endogenous gene product for either a cooperative factor or the normal target of the endogenous gene without elaborating the same activity, b) a poison pill (or monkey wrench) effect wherein the dominant negative gene product ACTIVELY interferes with a process required for normal gene function, c) a feedback effect, wherein the DN ACTIVELY stimulates a negative regulator of the gene function.

### Founder Animals, Animal Lines, Traits, and Reproduction

Founder animals (F0 generation) may be produced by cloning and other methods described herein. The founders can be homozygous for a genetic modification, as in the case where a zygote or a primary cell undergoes a homozygous modification. Similarly, founders can also be made that are heterozygous. The founders may be genomically modified, meaning that the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

In livestock, many alleles are known to be linked to various traits such as production traits, type traits, workability traits, and other functional traits. Artisans are accustomed to monitoring and quantifying these traits, e.g. Visscher et al., Livestock Production Science, 40:123-137, 1994; U.S. Pat. No. 7,709,206; U.S. 2001/0016315; U.S. 2011/0023140; and U.S. 2005/0153317. An animal line may include a trait chosen from a trait in the group consisting of a production trait, a type trait, a workability trait, a fertility trait, a mothering trait, and a disease resistance trait. Further traits include expression of a recombinant gene product.

### Recombinases

Embodiments of the invention include administration of a targeted nuclease system with a recombinase (e.g., a RecA protein, a Rad51) or other DNA-binding protein associated with DNA recombination. A recombinase forms a filament with a nucleic acid fragment and, in effect, searches cellular DNA to find a DNA sequence substantially homologous to the sequence. For instance a recombinase may be combined with a nucleic acid sequence that serves as a template for HDR. The recombinase is then combined with the HDR template to form a filament and placed into the cell. The recombinase and/or HDR template that combines with the recombinase may be placed in the cell or embryo as a protein, an mRNA, or with a vector that encodes the recombinase. The disclosure of U.S. 2011/0059160 (U.S. patent application Ser. No. 12/869,232) is hereby incorporated herein by reference for all purposes; in case of conflict, the specification is controlling. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assists in repair of DNA double strand breaks. RAD51 family members are homologous to the bacterial RecA and yeast Rad51. Cre recombinase is an enzyme that is used in experiments to delete specific sequences that are flanked by loxP sites. FLP refers to Flippase recombination enzyme (FLP or Flp) derived from the 2μ plasmid of the baker's yeast

Saccharomyces cerevisiae.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

### Compositions and Kits

The present invention also provides compositions and kits containing, for example, nucleic acid molecules encoding site-specific endonucleases, CRISPR, Cas9, ZNFs, TALENs, RecA-gal4 fusions, polypeptides of the same, compositions containing such nucleic acid molecules or polypeptides, or engineered cell lines. An HDR may also be provided that is effective for introgression of an indicated allele. Such items can be used, for example, as research tools, or therapeutically

sry (sex determining region Y) is a gene on the Y chromosome encoding a transcription factor. The gene is also known as testis determining factor (TDF). sry regulates the sox9 transcription factor which both increases the expression of sry and differentiation of sertoli cells, the effect of which is to promote differentiation of the genital ridge into testes rather than ovaries. Only mammals and marsupials (therians) use the XY sex determining system.

TABLE 1

| | sry mRNA length |
|---|---|
| Animal | Source |
| Mus musculus | 1188 |
| (house mouse) | NC_000087 |
| Homo sapiens | 887 |
| (human) | NM_003140 |
| Rattus norvegicus | 513 |
| (Norwedian rat) | GATN01000012 |
| Bos Taurus | 1040 |
| (cattle) | EU294189 |
| Sus scrofa | 711 |
| (pig) | NM_214452 |
| Equus caballus | 1403 |
| (horse) | NM_001081810 |
| Pan troglodytes | 910 |
| chimpanzee | AY679780 |
| Oryctolagus cuniculus | 624 |
| (rabbit) | NM_001171148 |
| Felis catus | 705 |
| (domestic cat) | NM_001009240 |
| Capra hircus | 723 |
| (goat) | Z30646 |
| Macaca mulatta | 615 |
| rhesus monkey | AF322901 |
| Ursus maritimus | 666 |
| polar bear | XM_008709959 |
| Bison bison | 690¹ |
| (bison) | XM_010860135 |
| Odobenus rosmarus divergens | 663 |
| Pacific walrus | XM_004417134 |
| Cervus elaphus | 614 |
| (red deer) | EF693906² |

¹ predicted
² exon 1

The present disclosure teaches introducing multiple copies of sry in the genome of a therian animal, when the animal is genotypically XX, the animal will be phenotypically male and sterile. Such animals can participate in agricultural uses just as a castrated animal without the expense of castration. Further, the XX male will not be available to mate with XY males thereby removing the animal from the reproductive potential of the population. In addition, XX sterile males will also compete for mating with true XX females. While the presence of XY males having multiple hereditable sry copies result in XX progeny being phenotypically male and thereby reducing the pool of fecund females, the XX males will also compete with the true XY males to mate with the remaining XX females. Therefore, in those populations, such as feral animals, that are in need of population control, this end can be achieved by 1) removing XX females from the population over a span of multiple generations and 2) having XY males compete with the overwhelming number of XX males that cannot produce viable offspring. Such means of population control would be very useful in the context of feral animals including, but not limited to pig, dog, cat and goat and the like, which cause tremendous damage when not controlled in the wild. In addition, such means of population control would also be amenable to use with wild pests such as mice, rats, and the like.

In addition, the inventors have also realized that, with respect to therian or livestock animals in agricultural use, such as castrated cattle (steers for docility and meat), goats, sheep, pigs, horses and the like, it may be desirable to limit the insertion of the sry gene to a single or several distinct loci on the genome. Such loci can be on any chromosome including somatic and sex chromosomes. This can be done with precision genome editing tools such as non-meiotic introgression of a zygote or a somatic cell by, such as, for example with tools such as using zinc finger nuclease, meganuclease, TALENs or CRISPR/CAS technology in addition to transposon systems as discussed above. In these embodiments, an animal can then be cloned using methods that are known in the art. (See, for example https://www.biotech.wisc.edu/services/transgenicanimal).

Briefly, after fertilization of an ovum by a male sperm, the male and female pronuclei have yet to fuse and are easily visible under a light microscope. Nucleic acid in a variety of conformations can be injected. These conformations include supercoiled DNA, ssDNA dsDNA, linear and circular, as well as RNA and protein can be injected into and be accepted by either the male or the female pronuclei. The oocytes are then transferred to the uterus of a pseudopregnant recipient. This method results in random and multiple sites of insertion of the DNA.

Other methods include the injection of embryonic stem cells into blastocyst stage embryos. This technique uses embryonic stem cells which are removed from a blastocyst. The cells are transfected by any convenient method known to the art (see above) and the can be implanted in an enucleated uterus of a pseudopregnant recipient.

Of course, those of skill in the art will appreciate that when a somatic cell has been transformed with at least one sry allele, an animal can be raised by somatic cell nuclear transfer, as is known in the art. Briefly, once the genome of a somatic cell has been transfected with an sry gene by any of the known (or as yet unknown) methods including but, not limited to, nonmeiotic introgression gene editing using zinc finger nuclease, meganuclease, TALENs or CRISPR/CAS technology, retroviral mediated gene transfer or the like, the nucleus from the somatic cell can be implanted in an enucleated oocyte to provide a zygote and implanted in a pseudopregnant recipient.

In addition, the disclosure teaches the insertion of multiple sry genes in the genome of an animal. While the expression of multiple sry alleles may be useful in agricultural animals, it can be especially beneficial in controlling animal populations. For example, multiple copies of sry can be specifically or randomly inserted into an animal's genome. Such multiple insertions can be made, specifically, using precision gene editing such as using zinc finger nuclease, meganuclease, TALENs or CRISPR/CAS technology, retroviral insertion, and the like, using various site specific targets to achieve nonmeiotic introgression. However, random insertions can be accomplished using transposon technology and pronuclear microinjection as is known in the art. These insertions can be hereditable.

For example, while most transposon systems known in the art useful for transfection of vertebrates can be used. Several are known for their proclivity for multiple and random insertions.

Such systems are commercially available such as, for example, including, but not limited to, the sleeping Beauty (SB system) commercially available from Discovery Genomics, Inc., the PiggyBac™ Transposon System, commercially available from System Biosciences, Inc., EZ-Tn5™ and HyerMu™ systems commercially available form Epicentre®.

Therefore, in another exemplary embodiment of the invention, one or many sry allele can be introduced into cells, zygotes, pronuclei and the like using transposons and/or microinjection to provide random and/or multiple insertion events. In this embodiment, two, three, four or more copies of sry can be inserted into an animal's genome. This is the Po generation. In this embodiment, XX animals will be phenotypically male and be sterile. XY animals will be phenotypically male and will not be sterile but will have at least one introduced copy of sry in their genome. In this embodiment, XY males having one or multiple sry alleles in their genome will be able to mate and provide hereditable sry alleles in one or multiple copies to an F1 offspring. When the F1 offspring are XX they will be phenotypically male and will be sterile. When the F1 offspring are XY they will be phenotypically male and fertile. The F1, XY offspring may continue to have hereditable sry alleles. Thus, when the F2 offspring are generated, XX offspring inheriting the sry allele will be phenotypically male and sterile. The F2 offspring generated that are XY will be phenotypically male and may continue to have hereditable sry alleles that can be contributed to an F3 generation. In this manner, populations of feral or wild animals can be controlled by both providing approximately 50% of the offspring that are sterile (XX males) but also because those sterile males will mate with females thereby limiting the availability of fecund females available to mate with fertile males. See, for example, Koopman et al., 1991 May 9;351(6322):117-21.

Further, other studies have shown that while sry protein is not well conserved, there are regions 5′ upstream that do show high homology with each other (Ross, D, 2008, BMC Molecular Biology). While these varied regions may represent the sry native promoter, in genomically modified animals, according to one exemplary embodiment disclosed here, the sry gene can be put behind an inducible promoter such as Hsp70.3, LAC, TRE or a constitutive promoter such as CMV, CaMV 35s, SV40, CMV, UBC, EF1A, PGK and CAGG.

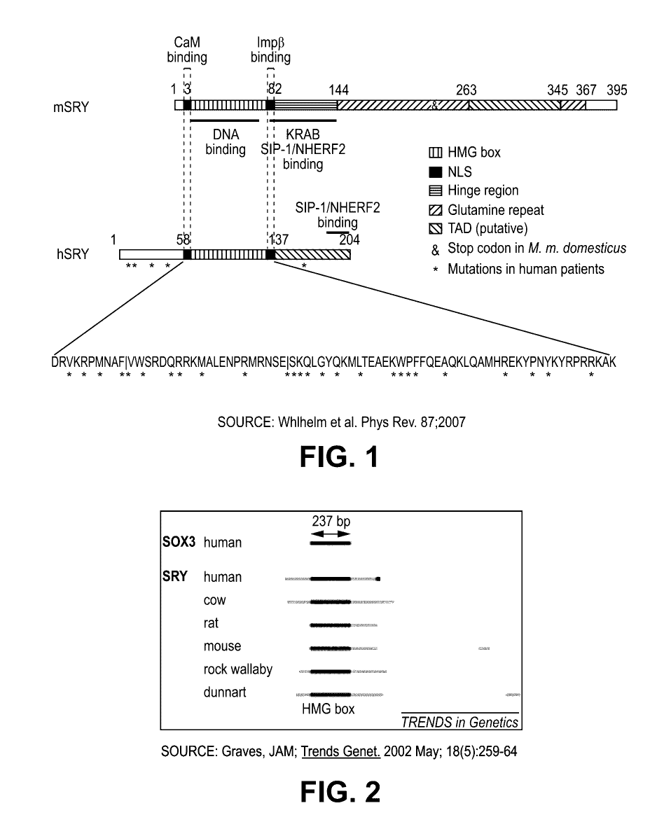

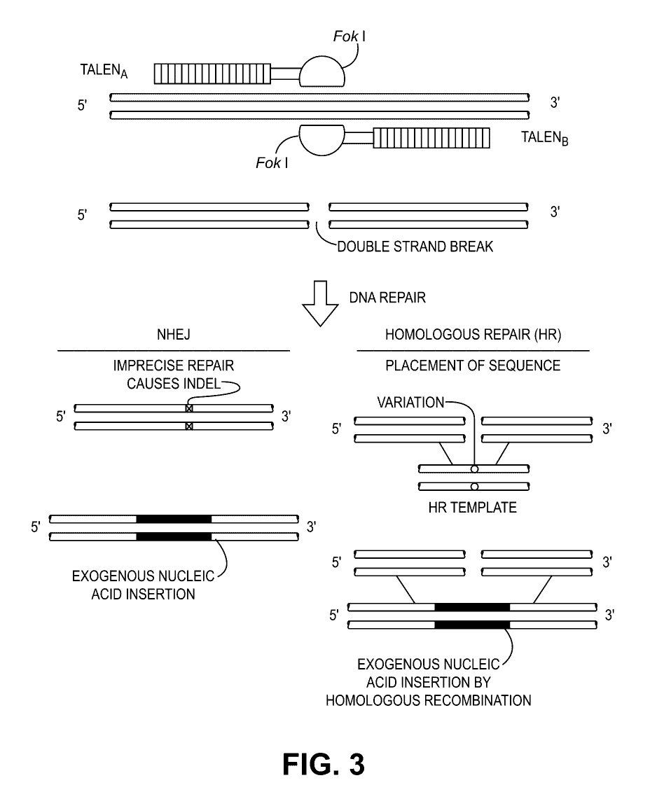

As illustrated in FIG. 1, there is a lack of homology of sry between species, with the exception of the HMG box. However, as shown by Pannetier et al. transgenic mice expressing goat sry, controlled by goat upstream sequences, resulted in XX mice having a male phenotype. Pannetier et al. Febs Lett 580(15) 2006. The HMG box binds to its cognate sequence on the minor groove of DNA inducing a 60-85° bend allowing transcription of its target sequence. It is unclear what the other regions of sry provide as their lack of homology with other species provides no guidance. The overall homology of sry and the sry DNA binding domain is further illustrated in FIG. 2.

Sry is dominant. When incorrectly expressed, it leads to gender reversal. For example, in humans, in rare instances (1 in 20,000), the sry allele translocates from the Y chromosome to the X chromosome where it results in an XX genotype of a phenotypical male termed “de la Chapelle Syndrome.” In most such cases, the masculinization is complete and no pathology is recognized until the subject undergoes treatment for sterility. According to one embodiment of the disclosure, when used to provide sterile males, such as, for example, to provide steers for the cattle industry, the gene is inserted into the genome using precision gene editing such as with gene editing using zinc finger nuclease, meganuclease, TALENs or CRISPR/CAS technology, retroviral insertion, and the like, using various site specific targets to achieve nonmeiotic introgression. There have also been reports of autosomal replication of the sry allele in men which is hereditable and responsible for sex reversal in the XX genotype. (Kasdan, R. et al., NEJM 1973, 288-539-545). The instant disclosure teaches exemplary embodiments in which sry is introduced into a single X chromosome and embodiments in which sry is introduced randomly throughout the genome in single or multiple copies.

Insertions into the X chromosome are known to those of skill in the art. See, for example, Wu et al. Neuron. 2014 Jan 8;81(1):103-19; incorporated herewith in its entirety. When an animal is cloned such as by somatic cell nuclear transfer (SCNT) or by pronuclear microinjection of a fertilized egg. The somatic cell can be taken from a male or a female and have either an XY or an XX genotype respectively, the fertilized egg may be either XX or XY. If the somatic cell is taken from a male and introgressed with sry no change in phenotype or fertility is expected. However, depending on the method and place of insertion, the resulting animal may have multiple sry copies inserted in the genome either in both the autosomes and the allosomes or only in the autosomes or the allosomes. In those cases where multiple copies of sry are inserted in the autosomes, the allele will segregate independently during meiosis (and mitosis). During gamete formation, if the haploid complement includes a copy of sry, offspring arising from fertilization by a sperm carrying the autosomal sry copy will result in autosomal expression of sry and will be phenotypically male regardless of its allosomal genotype. Further, if the offspring is genetically female e.g., having an XX genotype, the offspring will be sterile, e.g., a sterile, phenotypic male. Moreover, such animals can have normal sex drives (see, for example, Koopman et al., Nature 351, 117-121 (1991)) and regardless of the sterility of the animal, mated females generally form a vaginal plug after mating thereby limiting mating of the animal during that cycle. If the animal is genotypically male it will be fertile but the autosomal sry copies will segregate independently of the sex chromosomes. Therefore, if the offspring of the second generation is an XX female and receives an autosomal sry copy, that animal will be phenotypically male and sterile.

However, if the progeny of the male receives an X chromosome from the father, the progeny will inherit an X-linked sry allele resulting in a sterile, phenotypic male offspring. If the somatic cell is taken from a female, it will have an XX genotype and will be phenotypically female.

When sry is inserted into the X chromosome of a cell or embryo, it will be inserted into one or both X chromosomes. While numerous studies have shown that a single sry gene can drive gonadal differentiation to testes, in some embodiments, both X chromosomes have a sry insertion. Of course, those of skill in the art recognize that an XX sry male phenotype can be produced by introducing sry into any chromosome for expression. In various embodiments, a sterile XX male is generated by inserting sry at diverse sites throughout the genome.

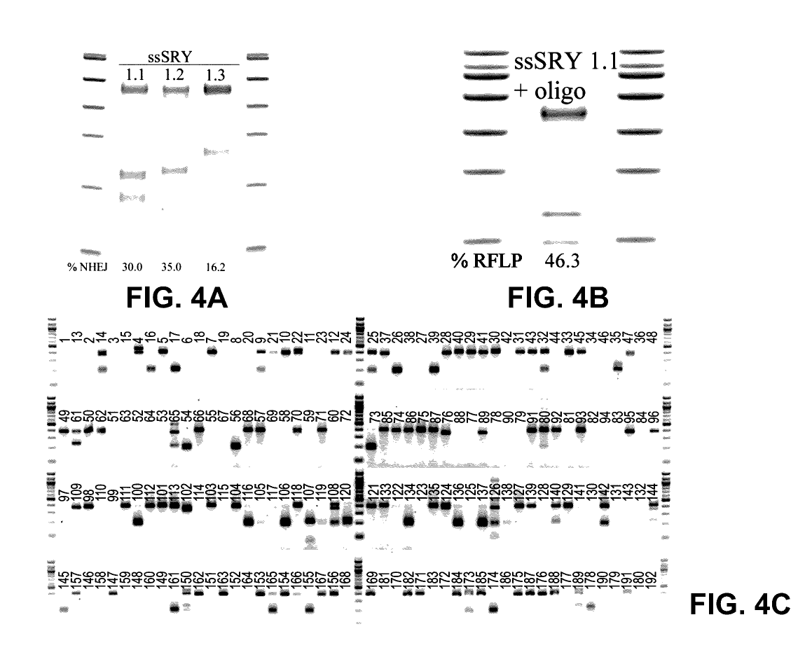

Similarly, a normal XY male can be modified to become a sterile phenotypic female by disrupting the sry gene on the Y chromosome. In this embodiment, TALENs, CRISPR/Cas, meganuclease and the like, specific for the conserved sequences of the HMG box are used. As illustrated in FIG. 3 by directly injecting TALEN specific nuclease into the one-cell embryo, pronucleus or somatic cell nucleus, a double-stranded break (DSB) can be generated in the genomic DNA at the site of the HMG box DSBs induced by TALENs or CRISPR/Cas mediated genome engineering will then be prepared by error-prone nonhomologous end joining (NHEJ) resulting in either a disruption in the sry reading frame or an sry protein that is unable to bind target sequences via its HMG box. The resulting animal will have an XY genotype but a sterile, female, phenotype. See, Hawkins, Hum Mutat. 1993;2(5):347-50

### EXAMPLE 1

### sry TALENs Design and Production

TALEN designing and production. Candidate TALEN target DNA sequences and RVD sequences are identified using the online tool “TAL Effector Nucleotide Targeter” (https://tale-nt.cac.cornell.edu/about). Plasmids for TALEN DNA transfection or in vitro TALEN mRNA transcription are then constructed by following the Golden Gate Assembly protocol using pC-GoldyTALEN (Addgene ID 38143) and RCIscript-GoldyTALEN (Addgene ID 38143) as final destination vectors (Carlson, 2013 PNAS). The final pC-GoldyTALEN vectors are prepared by using PureLink® HiPure Plasmid Midiprep Kit (Life Technologies) and sequenced before usage. Assembled RCIscript vectors prepared using the QIArep Spin Miniprep kit (Qiagen) were linearized by SacI to be used as templates for in vitro TALEN mRNA transcription using the mMESSAGE mMACHINE® T3 Kit (Ambion) as indicated previously. Modified mRNA was synthesized from RCIScript-GoldyTALEN vectors as previously described substituting a ribonucleotide cocktail consisting of 3′-0-Me-m7G(5′)ppp(5′)G RNA cap analog (New England Biolabs), 5-methylcytidine triphosphate pseudouridine triphosphate (TriLink Biotechnologies, San Diego, Calif.) and adenosine triphosphate and guanosine triphosphate. Final nucleotide reaction concentrations are 6 mM for the cap analog, 1.5 mM for guanosine triphosphate, and 7.5 mM for the other nucleotides. Resulting mRNA was DNAse treated prior to purification using the MEGAclear Reaction Cleanup kit (Applied Biosciences). Table II provides a list of TALEN that bind the coding sequence of sry providing a sry knockout.

TABLE II

| | | | | | Sense strand binding |
|---|---|---|---|---|---|
| | | | Left Monomer RVD | Right Monomer RVD | sequence-spacer is |
| Species | Gene | ID | sequence/SEQ ID NO. | sequence/SEQ ID NO. | lowercase/SEQ ID NO. |
| Sus | SRY | SsSRY | NN NI NI HD NN HD | NI NN NI NN HD HD | GAACGCTTTCATTGTG |
| Scrofa | | 1.1 | NG NG NG HD NI NG | NI HD NG NG NG NG | tggtctcgtgatcaaa |
| | | | NG NN NG NN/ | HD NG HD HD/ | GGAGAAAAGTGGCTCT |
| | | | SEQ ID NO. 26 | SEQ ID NO. 27 | SEQ ID NO. 28 |
| Sus | SRY | ssSRY | NI NN NI NN NI NI | NN HD NI NG HD HD | AGAGAACCCTCAAATG |
| Scrofa | | 1.2 | HD HD HD NG HD NI | HD NI NN HD HD NI | caaaactcagagatca |
| | | | NI NI NG NN/ | HD NG NG NN HD/ | GCAAGTGGCTGGGATGC/ |
| | | | SEQ ID NO. 29 | SEQ ID NO. 30 | SEQ ID NO. 31 |
| Sus | SRY | ssSRY | HD NI NN HD NI NI | NG NG NG HD NN | CAGCAAGTGGCTGGGA |
| Scrofa | | 1.3 | NN NG NN NN HD | NN HD NG NG HD | tgcaagtggaaaatgc |
| | | | NG NN NN NN NI/ | NG NN NG NI NI/ | TTACAGAAGCCGAAA/ |
| | | | SEQ ID NO. 32 | SEQ ID NO. 33 | SEQ ID NO. 34 |
| Bos | SRY | Bos | HD HD NN NG NN | HD NI HD NI NN HD | CCGTGTAGCCAATGTTA |
| taurus | | SRY | NG NI NN HD HD NI | NI NN HD NG NN NN | ccttattgtggcccag |
| | | 1.1 | NI NG NN NG NG NI/ | NI HD NI NI NN HD/ | GCTTGTCCAGCTGCTGTG/ |
| | | | SEQ ID NO. 35 | SEQ ID NO. 36 | SEQ ID NO. 37 |
| Bos | SRY | Bos | NG NN NG NN NN | NG HD NI HD NG HD | TGTGGCCCAGGCTTGTC |
| taurus | | SRY | HD HD HD NI NN NN | HD NG NN HD NI NI | cagctgctgtgatgct |
| | | 1.2 | HD NG NG NN NG | NI NI NN NN/ | CCTTTTGCAGGAGTGA/ |
| | | | HD/ | SEQ ID NO. 39 | SEQ ID NO. 40 |
| | | | SEQ ID NO. 38 | | |
| Bos | SRY | Bos | HD NG HD NG NN | NN NN NI NI NI NI | CTCTGTGCCTCCTCAAAG |
| taurus | | SRY | NG NN HD HD NG | NN NN HD NG NG NI | aatgggcgcttttcag |
| | | 1.3 | HD HD NG HD NI NI | HD NI NN NI NG NN/ | CATCTGTAAGCCTTTTCC/ |
| | | | NI NN/ | SEQ ID NO. 42 | SEQ ID NO. 43 |
| | | | SEQ ID NO. 41 | | |
| Bos | SRY | Bos | NG NN NI HD NN NG | HD NI NN NG NN NG | TGACGTGGTCCTGGCTG |
| taurus | | SRY | NN NN NG HD HD | NN NI NI NI NN NN | ctctccctaacatgtt |
| | | 1.4 | NG NN NN HD NG | NN NN NI NN/ | CTCCCCTTTCACACTG/ |
| | | | NN/ | SEQ ID NO. 45 | SEQ ID NO. 46 |
| | | | SEQ ID NO. 44 | | |

### Oligonucleotide Templates.

All oligonucleotide templates are synthesized by Integrated DNA Technologies, 100 nmole synthesis purified by standard desalting, and resuspended to 400 μM in TE. Table III provides a listing of TALEN characteristics. Table IV provides a list of PCR primers used.

TABLE III

| | | | | | | | | TALEN size and |
|---|---|---|---|---|---|---|---|---|
| | | | | | | | | repeat variable di- |
| | | | | % NHEJ | % NHEJ | | | residue sequence, |
| | | | ID/SEQ | D3, +231 | D3 GT | Spacer | | left TALEN shown |
| Organism | Gene | Exon | ID NO. | scaffold | scaffold | size | | in upper row |
| Sus Scrofa | sry | | sssry ds3.2/ | | 40 | 16 | 19 | NI NG NI HD |
| | | | SEQ ID | | | | | NI NG NG NG |
| | | | NO. 1 | | | | | NG NI HD NI |
| | | | | | | | | HD NI HD NI |
| | | | | | | | | NG NI NG |
| | | | SEQ ID | | | | 17 | NI NN NN NG |
| | | | NO. 2 | | | | | NG HD NI NN |
| | | | | | | | | NN HD HD NI |
| | | | | | | | | NG NG NI NI |
| | | | | | | | | NG |
| Sus Scrofa | p65 | 11 | p65_11-1/ | | 39 | 16 | 16 | NN HD HD HD |
| | | | SEQ ID | | | | | HD HD HD HD |
| | | | NO. 3 | | | | | NI HD NI HD |
| | | | | | | | | NI NN HD NG |
| | | | SEQ ID | | | | 16 | NI NG NI NN |
| | | | NO. 4 | | | | | HD HD NG HD |
| | | | | | | | | NI NN NN NN |
| | | | | | | | | NG NI HD NG |
| Sus Scrofa | DMD | 7 | DMD7.1L + | 17 | 38 | 15 | 18 | NN NN NI NI |
| | | | 7.1R/ | | | | | HD NI NG NN |
| | | | SEQ ID | | | | | HD NI NG NG |
| | | | NO. 5 | | | | | HD NI NI HD |
| | | | | | | | | NI NG |
| | | | SEQ ID | | | | 20 | HD HD NI NN |
| | | | NO. 6 | | | | | NG NI NN NG |
| | | | | | | | | NG NG HD NG |
| | | | | | | | | HD NG NI NG |
| | | | | | | | | NN HD HD NG |
| Sus Scrofa | sry | sssry | SEQ ID | | 20 | 15 | 18 | NI NG NI NG |
| | | ds3.3 | NO. 7 | | | | | NN NI NI NI |
| | | | | | | | | HD NG NN NI |
| | | | | | | | | HD NI NN NG |
| | | | | | | | | NI NG |
| | | | SEQ ID | | | | 19 | HD HD HD NI |
| | | | NO. 8 | | | | | NI NG HD NG |
| | | | | | | | | NN NI NN NG |
| | | | | | | | | NG HD NG NN |
| | | | | | | | | NN HD NG |

TABLE IV

| | | | SEQ |
|---|---|---|---|
| | | | ID |
| Target ID | Forward | Reverse | NO. |
| sssry ds3.2 | GCTCCTGGCCATCTCTTTG | TGCCTGCCTGCTTGCATCTC | 9/10 |
| | GTCA | TCA | |
| p65_11-1 | GCAATAACACTGACCCGAC | GCAGGTGTCAGCCCTTTAGG | 11/12 |
| | CGTG | AGCT | |
| DMD7.1L + 7.1 | GGAATATGGGCATGTGTTG | TGCAGTATACTTCATCCACG | 13/14 |
| R | TCAGTC | AGGCA | |

### EXAMPLE 2

### Tissue Culture and Transfection

Livestock fibroblasts are maintained at 37° C. or 30° C. (as indicated) at 5% CO₂ in DMEM supplemented with 10% fetal bovine serum, 100 I.U./ml penicillin and streptomycin, and 2mM L-Glutamine. For transfection, all TALENs, CRISPR/Cas9 and HDR templates are delivered through transfection using the Neon Transfection system (Life Technologies) unless otherwise stated. Briefly, low passage bovine fibroblasts reaching 100% confluence were split 1:2 and harvested the next day at 70-80% confluence. Each transfection is comprised of 500,000-600,000 cells resuspended in buffer “R” mixed with mRNA and oligos and electroporated using the 100 ul tips by the following parameters: input Voltage; 1800V; Pulse Width; 20 ms; and Pulse Number; 1. Typically, 0.1-5 of TALEN mRNA and 2-5 μM of oligos specific for the sry mutation desired are included in each transfection along with oligos entering the required restriction site for RFLP analysis. After transfection, cells are divided 60:40 into two separate wells of a 6-well dish for three days' culture at either 30° C. or 37° C. respectively. After three days, cell populations are expanded and at 37° C. until at least day 10 to assess stability of edits.

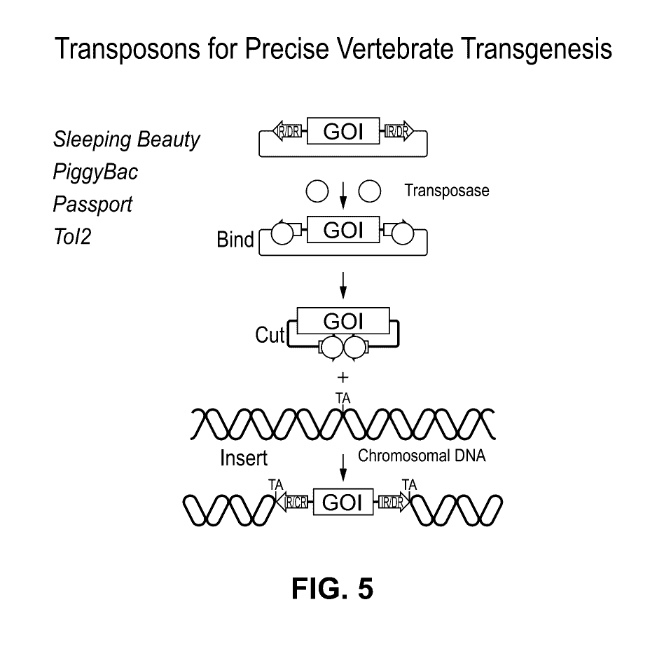

To disrupt porcine SRY, three pairs of TALENs, ssSRY1.1, ssSRY1.2 and ssSRY1.3 (Table II), were developed that target the coding sequence of SRY in EXON 1. When transfected into swine fibroblasts, each TALEN pair displayed activity at day 3 of 30, 35 and 16.2 percent NHEJ, respectively as measured with a Surveyor Nuclease assay (FIG. 4A). TALEN pair ssSRY1.1 was selected from this group for production of SRY KO fibroblast colonies by HDR with an oligonucleotide template (cgtgtcaagcgacccatgaacgctttcattgtgtggtctcgtTAAGCTTgatcaaaggagaaaagtggctctagagaaccctcaaatgca) (SEQ ID NO. 47). The ssODN was designed to introduce a premature termination codon (italicized text) and a novel HindIII restriction site (underlined) for RFLP genotyping. When transfected into male pig fibroblasts with the ssSRY1.1 TALEN pair, RFLP genotyping of day 3 populations revealed introgression of 46.3% (FIG. 4B).

Dilution cloning: Three days post transfection, 50 to 250 cells are seeded onto 10 cm dishes and cultured until individual colonies reached circa 5 mm in diameter. At this point, 6 ml of TrypLE (Life Technologies) 1:5 (vol/vol) diluted in PBS was added and colonies were aspirated, transferred into wells of a 24-well dish well and cultured under the same conditions. Colonies reaching confluence were collected and divided for cryopreservation and genotyping. Colonies generated from the ssSRY1.1+ssODN population were screened by RFLP assay using HindIII (FIG. 4C). Several colonies genotyped positive for introgression of the ssODN; i.e. 16, 17, 36 whereas some genotyped as wild type, i.e. 28, 29, 30. Others show evidence of large deletions via NHEJ; i.e. 76, 102 that would disrupt SRY function. These clones where SRY is disrupted by TALENs are used to generate founder animals by cloning.

### EXAMPLE 3

### Surveyor Mutation Detection and RFLP Analysis

Sample preparation: Transfected cells populations at day 3 and 10 are collected from a well of a 6-well dish and 10-30% were resuspended in 50 μl of 1X PCR compatible lysis buffer: 10 mM Tris-Cl pH 8.0, 2 mM EDTA, 0.45% Triton X-100(vol/vol), 0.45% Tween-20(vol/vol) freshly supplemented with 200 μm/ml Proteinase K. The lysates were processed in a thermal cycler using the following program: 55° C. for 60 minutes, 95° C. for 15 minutes. Colony samples from dilution cloning were treated as above using 20-30 μl of lysis buffer.

PCR flanking the intended sites is conducted using Platinum Taq DNA polymerase HiFi (Life Technologies) with 1 μl of the cell lysate according to the manufacturer's recommendations. The frequency of mutation in a population is analyzed with the Surveyor Mutation Detection Kit (Transgenomic) according to the manufacturer's recommendations using 10 ul of the PCR product as described above. RFLP analysis is performed on 10 μl of the above PCR reaction using the indicated restriction enzyme. Surveyor and RFLP reactions are resolved on a 10% TBE polyacrylamide gels and visualized by ethidium bromide staining. Densitometry measurements of the bands is performed using ImageJ; and mutation rate of Surveyor reactions are calculated as described in Guschin et al. 2010(4). Percent HDR is calculated via dividing the sum intensity of RFLP fragments by the sum intensity of the parental band +RFLP fragments. For analysis of restriction site incorporation, small PCR products spanning the target site were resolved on 10% polyacrylamide gels and the edited versus wild type alleles could be distinguished by size and quantified. RFLP analysis of colonies is treated similarly except that the PCR products are amplified by 1×MyTaq Red Mix (Bioline) and resolved on 2.5% agarose gels.

### EXAMPLE 4

### Production of Animal Clones Expressing sry Mutations

Upon confirmation of the stable sry mutations described above in a swine genome, somatic cell nuclear transfer or pronuclear microinjection, can be used to produce a cloned animal carrying the mutation. Briefly, a transgenic swine cell (or other artiodactyl if desired) such as an embryonic blastomere, fetal fibroblast, adult fibroblast, or granulosa cell that includes a nucleic acid mutation described above, is introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed “eggs”. After producing a bovine (or other artiodactyl) embryo (e.g., by fusing and activating the oocyte), the embryo is transferred to the oviducts of a recipient female, about 20 to 24 hours after activation or up to 8 days after activation in cattle. See, for example, Cibelli et al. (1998) Science 280, 1256-1258 and U.S. Pat. No. 6,548,741. Recipient females can be checked for pregnancy starting at 17 days after transfer of the embryos.

### EXAMPLE 5

### CRISPR/Cas9 Design and Production

Gene specific gRNA sequences were cloned into the Church lab gRNA vector (Addgene ID: 41824) according their methods (Mali, 2013). The Cas9 nuclease was provided either by co-transfection of the hCas9 plasmid (Addgene ID: 41815) or mRNA synthesized from RCIScript-hCas9. This RCIScript-hCas9 was constructed by sub-cloning the XbaI-AgeI fragment from the hCas9 plasmid (encompassing the hCas9 cDNA) into the RCIScript plasmid. Synthesis of mRNA was conducted as above except that linearization was performed using KpnI.

### EXAMPLE 6

### Transposons Allow For Precise Vertebrate Transgenesis

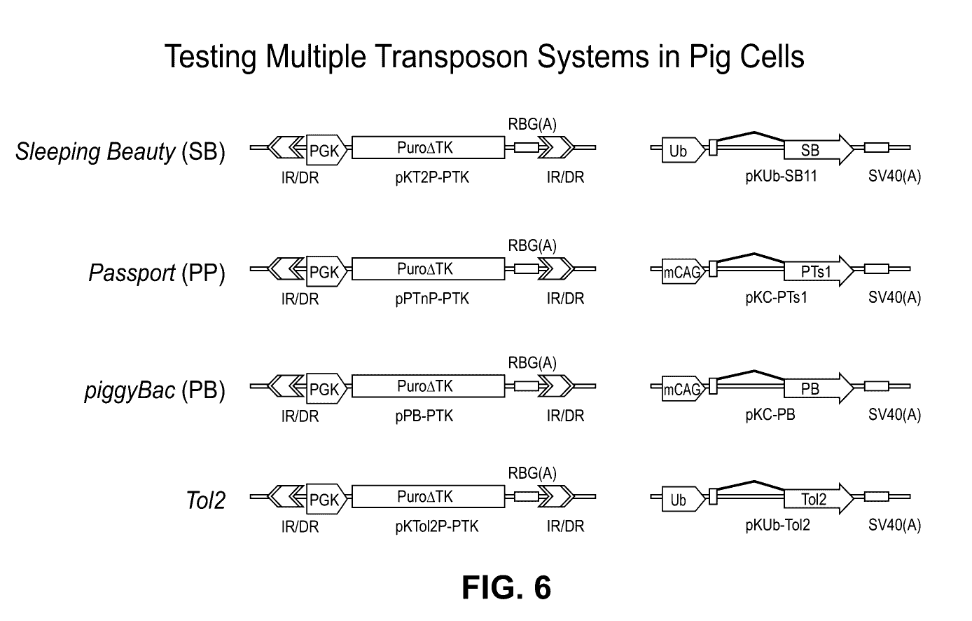

Transposon systems allow for the insertion of precisely defined DNA sequences into the chromosome of vertebrate animals. Such transposon systems include, but are not limited to, the Sleeping Beauty Transposon™ System (Discovery Genomics, Inc.); piggyBac™ Transposon System (Transposagen Biopharmaceuticals, Inc.); Passport Transposon System; Frog Prince Transposon System; and Tol2. Transposon systems such as described herein include a transposase and a transposon. The transposon is identified by the mirrored sets of nucleotide sequences, inverted repeats (IR) and direct repeats (DR) which define the boundaries of the transposon and surround the transposable sequence or the gene of interest (GOI), FIG. 5. The transposase binds to the IR/DRs and cuts the transposon out of a plasmid or nucleotide construct. The transposase inserts the transposon into a thymine/adenine (TA) base pair. As discussed above, the transposase can be provided as a protein, encoded on the same nucleic acid construct as the target nucleic acid, can be introduced on a separate nucleic acid construct, or provided as an mRNA (e.g., an in vitro-transcribed and capped mRNA).

### EXAMPLE 7

### Multicopy Transgenesis in Pig Cells

The efficiency of colony formation using several transposon systems was examined in fetal pig fibroblasts by co-transfection of the puroΔtk transposon (FIG. 6, left) in the identified transposon systems with transposase expression constructs (FIG. 6, right). The selectable marker puroAtk is a fusion protein of puromycin N-acetyl-transferase (Puro) and thymidine kinase under control of a phosphoglycerate kinase promoter, which enables positive selection in puormycin and negative selection in ganciclovir.

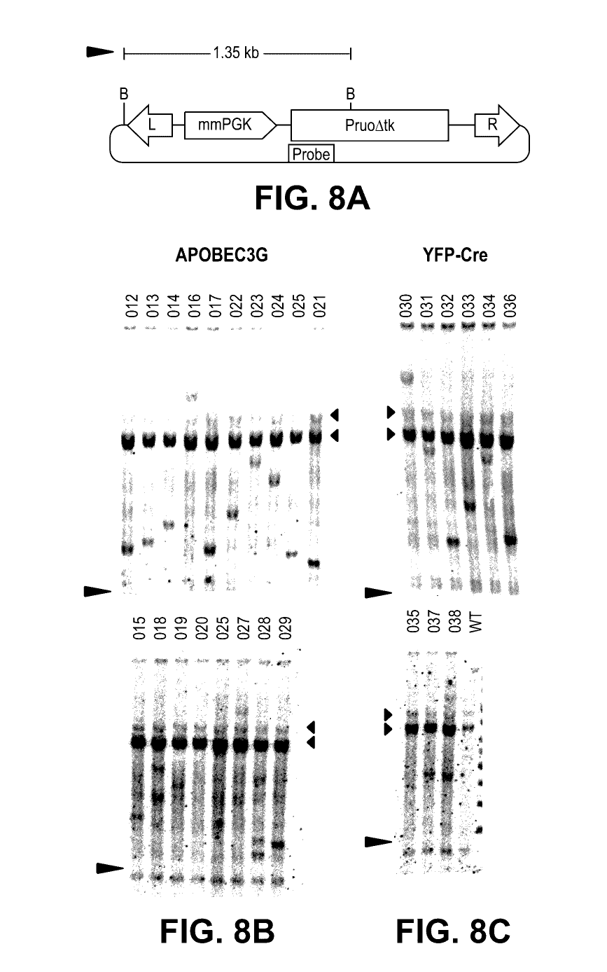

FIGS. 7A-7I shows the Activity of multiple transposon systems in PEGE cells. 7A) A drawing of a generic transposon (pTP-PTK) used for colony formation assays. The transposons used, except the transposon-specific inverted terminal repeats, are identical. The vector backbones of the transposons are also identical except for pGTol2P-PTK. The pKx-Ts drawing is a generic representation of the transposase-expressing vector. The promoter choices include Ub, CMV, and mCAGs for SB, Tol2, and PB and PP, respectively. The vector backbones and poly(A, pA) signals are identical except for pCMV-Tol2; 7B-7E) The number of colonies formed with SB, PP, Tol2, or PB PTK transposons are shown with βgal instead of transposase (−Ts) and with transposase (+Ts), where Ts is SB, PP, Tol2, or PB. In each case, the significance of transposase was verified with an unpaired t-test (p-values ≦0.00002). The PTK cassette served as both an SM and transcriptional termination by the inclusion of three poly A (pA) signals. 7F-7I) Southern blot of PEGE Clones. Individual puromycin resistant PEGE colonies were isolated and expanded for Southern analysis: 7F) Tol2; 7G) PB; 7H) SB; 7I) PP. Each transposon donor plasmid transfected into PEGE cells is diagrammed with restriction endonuclease sites used for DNA digestion and the probe fragment indicated (diagonal lined rectangle). Expected concatemer sizes (vertical lined arrow)/smallest possible transposition event (open arrow) for each transposon are 5159/3335 bp; 5083/3275 bp; 6285/3346 bp; and 5140/3320 bp, respectively. The positions of the marker bands are indicated by black dots on the right of each blot with sizes of 12, 10, 8, 6, 5, 4 and 3 kb are shown. Identification of multiple transposational events is shown by arrow marked “Jackpot”.

### EXAMPLE 8

### Marker-Free Transgenesis Using Transposons

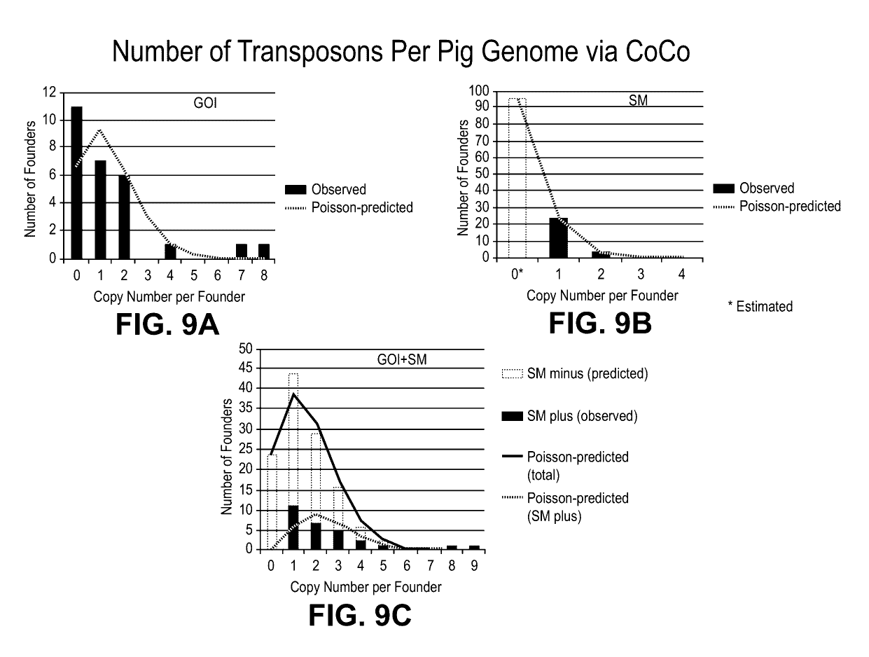

FIGS. 8A-8C show a Southern analysis of SM copy number. 8A) the Pkt2p-PuroΔtk transposon was used for selection of APOBEC3G and YFP-Cre cells by co-transpositional co-selection (CoCo). BamHI digestion of genomic DNA from both APOBEC3G (8B) and YFP-Cre (8C) founders would result in a 1.35-kb band (large black arrow) in animals harboring a concatemer insertion while transposase-mediated events are evident as slower-migrating fragments. The location of the probe fragment for PuroΔtk is shown in the diagram. The majority of APOBEC3G (8B) and YFP-Cre (8C) founders harbor only one copy of the SM. Two additional bands are also apparent in each animal (small double arrows). These bands are also apparent in a wild type control and correspond to hybridization to a repetitive element (RE).

### EXAMPLE 9

### Co-Transpositional Co-Selection (CoCo).

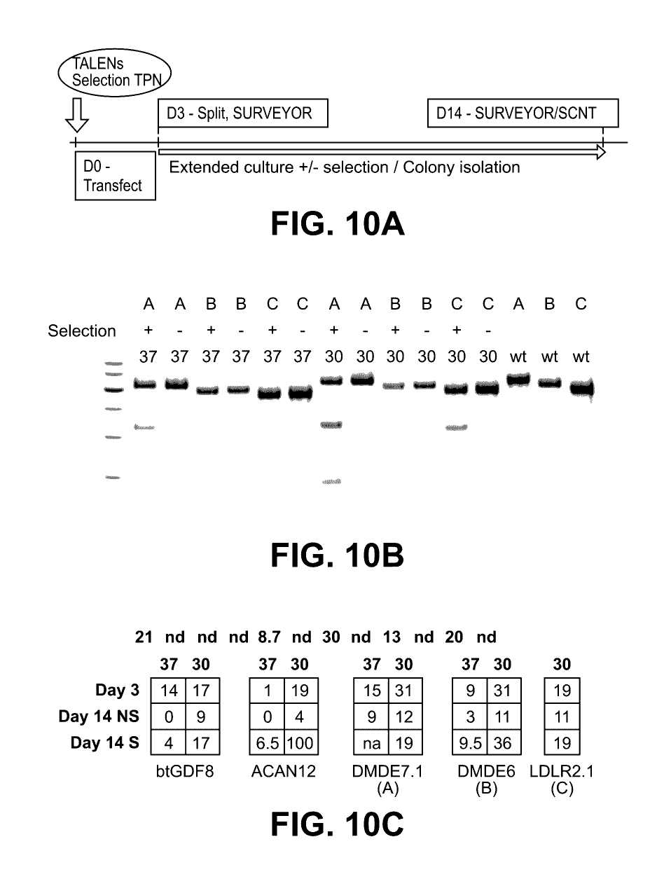

FIG. 9: Transgene copy number distribution in pigs and donor cells using co-transpositional co-selection (CoCo). 9A) 16 out of 27 (59%) pigs have at least one GOI (gene of interest) insertion, and the average insertion rate is 1.4 GOI insertions per founder. The observed distribution of GOI copies per founder is shown as filled bars. The upper curve (line) shows the copy number distribution predicted by a Poisson distribution with a mean of 1.4. The observed frequencies from the limited sampling roughly correspond to predicted, although pigs with zero copies are higher than expected, and pigs with more than 5 copies were not predicted. 9B) All founders carry at least one SM, as transfected cells were selected for antibiotic resistance prior to cloning. Therefore the average SM insertion rate in cells cannot be directly calculated because the frequency of transgenic cells lacking SM before elimination by selection is unknown. However, the average SM insertion rate can be estimated by fitting the observed data to a Poisson distribution. The filled bars show the observed SM insertions. The white (unfilled) bar corresponds to the number of pigs that could have been cloned if transgenic cells lacking SM were not eliminated by selection. This predicts that prior to selection 78% of transgenic cells lacked SM, consistent with the transfected molar ratio of GOI:SM at 4:1. The upper curve (line) shows the corresponding Poisson distribution of SM copy number with a best-fit average of 0.25 per founder. (9C) The sum of GOI and SM inserts in pigs should follow a Poisson distribution with a mean insertion number equal to the sum of the GOI and SM means (1.4+0.25=1.65), illustrated by the upper curve (line). However, since animals from SM minus cells could not be observed, the full distribution of inserts is unknown. SM-minus and SM-plus cells should contain the same distribution of GOI inserts because SM and GOI insertion events are independent, therefore GOI insert distribution in SM-minus cells was estimated based on a Poisson distribution with a mean 1.4. The filled columns show the distribution of GOI+SM inserts in SM-plus founders, whereas the stacked white columns show the predicted GOI+SM in SM-minus cells. The distribution of GOI+SM (stacked columns) generally tracks with the Poisson distribution. Finally, subtracting the unobserved distribution from the estimated total distribution predicts the GOI+SM distribution in SM containing cells lower curve (line), which matches well the observed distribution in transgenic pigs (filled columns), although the number of pigs with a single transgene was double what was expected.

### EXAMPLE 10

### Transposon Transposition And Introgression Coselction

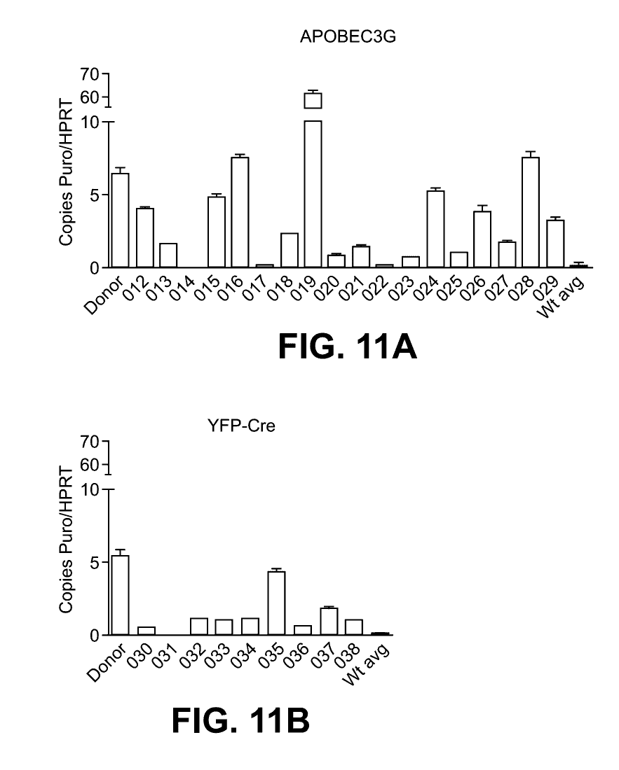

Transposon coselection for indel enrichment is shown in FIG. 10. In this experiment, cells were co-transfected with both transposon transposition and TANENs introgression. 10A) The experimental timeline. Day zero (D0), cells were transfected with a mixture of plasmids, including an expression cassette for each TALEN, a transposon encoding a selection marker, and transposase expression cassette. Transfected cells were cultured for 3 d at either 30 or 37° C. before splitting, collection of a sample for Surveyor assay, and replating for extended culture with and without selection for transposon integration. Cells cultured for 14+d were collected for Surveyor assay. 10B) Fibroblasts were transfected using Mirus LT1 reagent and Surveyor assay was performed on day 14 populations. Temperature treatment, selection and TALEN identification (identified by single letters (A, B, and C) as indicated in 10C are shown above the gel. 10C) Fibroblasts were transfected by nucleofection and the percent NHEJ was measured at day 3, and in day 14 nonselected (NS) and selected (S) populations. Temperature treatment is indicated above each matrix. ND, not detected; WT, wild-type amplicon, Surveyor-treated. This experiment shows the viability of co-transfection with transposon transposition and allele modification with non-meiotic introgression to both interrupt the native sry allele and/or add it back in trans.

### EXAMPLE 11

### SM Expression in APOBEC3G and YFP-Cre Founders.

FIG. 11 shows SM expression (PuroΔtk) in tails of APOBEC3G (11A) and YFP-Cre (11B) founders. Expression levels and standard deviations were determined by comparing the average of 3 replicate qPCR reactions to a standard curve generated from known templates and are reported as the ratio puro to HPRT copies. Levels of PuroΔtk observed in tail biopsy were similar to that observed in the transgenic donor cells (Donor) used for cloning.

### EXAMPLE 12

### The Economics Of Pig Transpositional Transgenesis

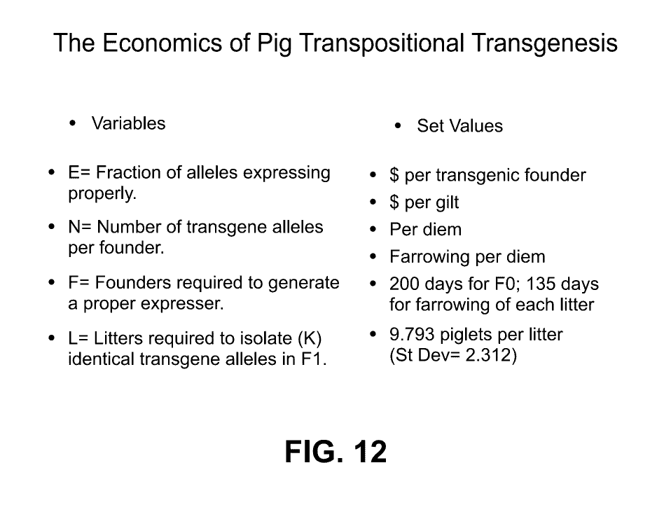

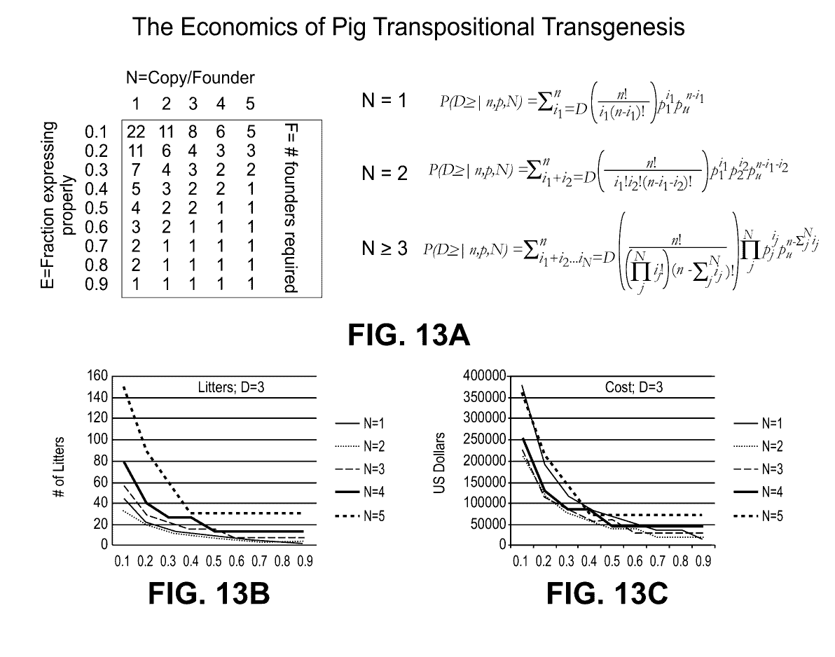

The economics of pig transpositional transgenesis (TnT) is illustrated in FIG. 12. Shown are the variables to be considered when determining the efficacy of inserting multiple alleles in a founder. FIG. 13 further investigates the variables identified in FIG. 12.

FIG. 13, the ability of TnT to introduce multiple unlinked transgene loci represents a significant enhancement over standard cellular transgenesis: cloning of pigs, minimizing the number of founders that need to be generated by providing multiple transgene loci that can be segregated and subjected to expression analysis in subsequent generations. The value of multi-loci founders has been modeled based primarily on two independent variables, the number of transgene insertions and the percentage of transgenes expected to express appropriately (13A). The number of transgenes per founder (N) can be controlled by manipulating the transposon system. The percentage of properly expressing transgene loci (E), however, is subject to intrinsic features of the transgene, as well as extrinsic features of the genome (position effects). The interaction of these two parameters, N and E, influences the number of founders (F) required to ensure the presence of a properly expressing locus. With 90 percent confidence, F was calculated over a range of N and E, where F=1 n(1-.9)/(N 1 n(1-E)).

FIG. 13A) displays the total number of founders required to have 90 percent confidence in capturing a properly expressing transgene locus over a range of proper expression (E) and number of transgenes (N). As anticipated, the number of founders required increases significantly at low values of E and N. While a high N value reduces the number of founders required to ensure a properly expressing locus, it concurrently raises the complexity of loci segregation in the F1 generation. Therefore, a second consideration of the model is to determine how many litters (L) are needed to produce the desired number of offspring with a distinct isolated locus (D) for expression analysis and line propagation. Assume all loci are unlinked, hemizygous for the insertion, and each locus will segregate according to Mendelian genetics (insert vs null). Given that a founder has inserts at N loci, there are 2^{N} possible gametes, of which N will carry only 1 insert each with probability pᵢ=½^{N}, all other gametes will carry either no, or multiple copies, for screening these are considered the undesirable type gametes. The probability of an undesirable gamete type is pᵤ=(1−N/2^{N}). Thus, for N inserts in a given founder, the probability that n offspring will produce at least D individuals heterozygous for only the i^{th} insert for each of the N loci is: For 1 insert the distribution of genotypes follows a binomial with

$$P ( D \geq \vert n , p , N ) = {\sum }_{{i}_{1} = D}^{n} ( \frac{n !}{{i}_{1} ( n - {i}_{1} ) !} ) {p}_{1}^{{i}_{1}} {p}_{u}^{n - {i}_{1}} ,$$

for 2 inserts a trinomial distribution with

$$P ( D \geq \vert n , p , N ) = {\sum }_{{i}_{1} + {i}_{2} = D}^{n} ( \frac{n !}{{i}_{1} ! {i}_{2} ! ( n - {i}_{1} - {i}_{2} ) !} ) {p}_{1}^{{i}_{1}} {p}_{2}^{{i}_{2}} {p}_{u}^{n - {i}_{1} - {i}_{2}} ,$$

and for N inserts a multinomial

distribution, with

$$P ( D \geq \vert n , p , N ) = {\sum }_{{i}_{1} + {i}_{2} + … {i}_{N} = D}^{n} ( \frac{n !}{( {\prod }_{j}^{N} {i}_{j} ! ) ( n - {\sum }_{j}^{N} {i}_{j} ) !} ) {\prod }_{j}^{N} {p}_{j}^{{i}_{j}} {p}_{u}^{n - {\sum }_{j}^{N} {i}_{j}}$$

Numerical methods can be used to determine the minimum n required to achieve at least D offspring with the same genotype at a given probability threshold, here set to 0.9. Average litter size was set to 9.793 pigs per litter with a standard deviation of 2.312(4). Since the average litter size and standard deviation are from a large data set, we can assume a normal distribution and use the standard Z-value for a chosen probability threshold (Z=1.282 at 90% probability). Using numerical methods, the number of litters required per founder (L) can be determined using

$$n = L ( - 1.282 ( \frac{2.312}{\sqrt{L}} ) + 9.793 .$$

(13B) The total number of litters (Lₜₒₜₐₗ) required to isolate D=3 identical, transgene loci for each expression threshold can be found by multiplying the number of founders required by litters per founder. As expected, more litters are required with greater number of inserts per founder. However, an elevated number of F1 litters can be offset by savings in founder generation. (13C) An economic model based on current costs for each component of transgenic line generation was developed. Commercial cloning services are currently available at retail pricing of about $10,000 dollars per founder. Maintaining male clones to sexual maturity (200 days) requires per diems of $2500 each ($12.50/day) at University of Minnesota rates. The expense of outcrossing includes gilts (estimated at $300 each), as well as per diems during gestation, through farrowing and weaning ($1991, 135 days at $14.75/day) is also included. The estimated cost of developing a transgenic line for a given expression frequency and founder copy number is equal to F($10,000)+F($2500)+Lₜₒₜₐₗ($300)+Lₜₒₜₐₗ ($1991). Generation of founders with 2-4 independent transgenes consistently provides the lowest cost, which becomes more significant as E drops below 60%.

### EXAMPLE 13

### Transposon Resident sry For Creating Daughterless Boars For Feral Pig Elimination

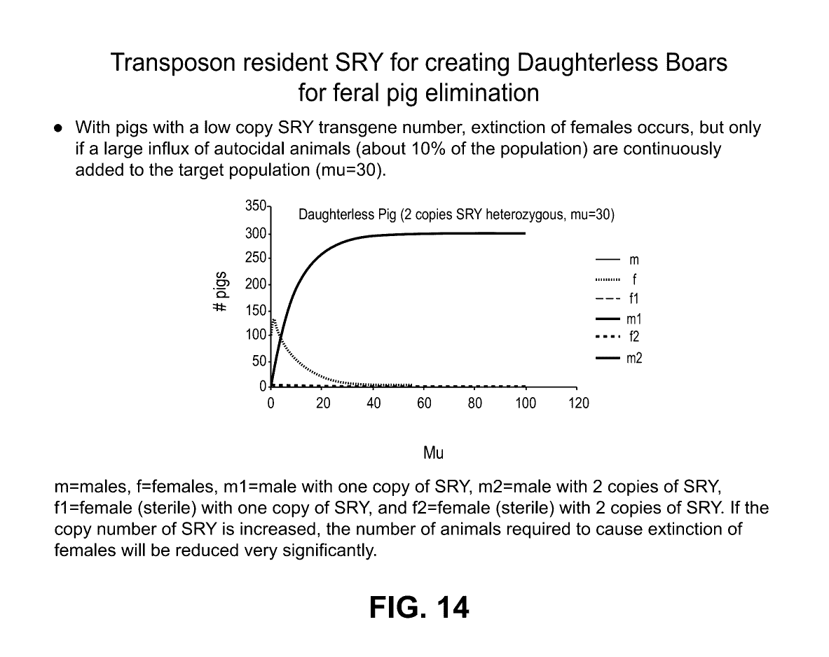

With pigs with a low copy sry transgene number, extinction of females occurs, but only if a large influx of autocidal animals (about 10% of the population) are continuously added to the target population. FIG. 14 illustrates graphically that as the number of males with at least two transgenes is kept high, the number of females (and other animals) will approximate zero. Thus, the ability to insert multiple copies of sry will allow faster extinction of feral animals in a population. Thus, as shown in FIGS. 13A-C and as calculated in the functions discussed in Example 12, the calculation of the chance that an offspring will not get the sry transposon is 0.5N where N is the copy number. Thus, if the male carrier has 10 sry transgenes there is a 0.1 percent chance of getting an F1 without the sry transgene. In such instances, were the number of transgenes high per offspring, the number of offspring inheriting the sry transgenes in following populations can be maintained high relative to a low copy number of transgenes that may or may not be hereditable.

While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this invention, as set forth above, are intended to be illustrative not limiting. Various changes may be made without departing from the spirit and scope of the invention. Therefore, the invention is intended to embrace all known or later-developed alternatives, modifications, variations, improvements and/or substantial equivalents of these exemplary embodiments.

The following paragraphs enumerated consecutively from 1 through 94 provide for various additional aspects of the present invention. In one embodiment, in a first paragraph:

1. A non-human animal having expressed in its genome an introduced nucleic acid comprising one or more sry alleles.

2. The non-human animal of paragraph 1, wherein the animal has an X and a Y chromosome and is phenotypically male.

3. The non-human animal of paragraphs 1 or 2, wherein the animal has two X chromosomes and is phenotypically male.

4. The non-human animal of paragraphs 1 through 3, wherein the animal is sterile.

5. The non-human animal of paragraphs 1 through 4, wherein at least one introduced sry allele is on one or more X chromosomes.

6. The non-human animal of paragraphs 1 through 5, wherein at least one introduced sry allele is on one or more autosomes.

7. The non-human animal of paragraphs 1 through 6, wherein the one or more introduced sry alleles are introduced by nonmeiotic introgression.

8. The non-human animal of paragraphs 1 through 7, wherein the introduced sry alleles are hereditable.

9. The non-human animal of paragraphs 1 through 8, wherein the animal is a livestock animal.

10. The non-human animal of paragraphs 1 through 9, wherein the offspring have two X chromosomes and are phenotypically male.

11. The non-human animal of paragraphs 1 through 10, wherein the offspring are sterile.

12. The non-human animal of paragraphs 1 through 11, wherein the introduced allele is an ortholog.

13. The non-human animal of paragraph 1 through 11, wherein the introduced allele is a paralog.

14. A non-human animal comprising a genomic modification of an HMG box of a sry allele.

15. The non-human animal of paragraph 14, wherein the animal has an X and a Y chromosome and is phenotypically female.

16. The non-human animal of paragraphs 14 and 15, wherein the animal is sterile.

17. The non-human animal of paragraphs 14 through 16, wherein the animal is a livestock animal.

18. The non-human animal of paragraphs 14 through 17, wherein the modification results in a break in protein synthesis of the gene.

19. The non-human animal of paragraphs 14 through 18, wherein the modification is an insertion or deletion.

20. The non-human animal of paragraphs 14 through 19, wherein the modification results in an inability of sry protein to bind to its target DNA.

21. The non-human animal of paragraphs 1-20, wherein the one or more sry alleles are introduced by transposon transposition.

22. The non-human animal of paragraphs 1-21, wherein the transposon system includes

Sleeping Beauty, Passport, Frog Prince, Tol2, or PiggyBac.

23. The non-human animal of paragraphs 1-22, wherein the one or more sry alleles is introduced into the genome without the use of selectable markers.

24. The non-human animal of paragraphs 1-23, wherein the animal is coselected for transposon transposition and non-meiotic introgression.

25. The non-human animal of paragraphs 1-24, wherein non-meiotic introgression interrupts the native sry allele.

26. The non-human animal of paragraphs 1-25, wherein transposon transposition provides multiple insertions of the sry allele.

27. The non-human animal of paragraphs 1-26, wherein the animal is a cow, pig, chicken, goat, sheep, dog, cat, rodent, deer.

28. A method for providing a sterile, phenotypically male non-human animal comprising: introducing one or more sry alleles into the genome of an animal having an XX genotype

29. The method of paragraph 29, wherein the one or more sry alleles are introduced into an X chromosome.

30. The method of paragraphs 28 and 29, wherein the one or more sry alleles are introduced into both X chromosomes.

31. The method of paragraphs 28 through 30, wherein the one or more sry alleles are introduced into one or more autosomes.

32. The method of paragraphs 28 through 31, wherein the one or more sry alleles are introduced into the nucleus of a somatic cell.

33. The method of paragraphs 28 through 32, wherein the one or more sry alleles are integrated into the genome by nonmeiotic introgression.

34. The method of paragraphs 28 through 33, wherein the one or more sry alleles are introduced into the genome using CRISPR/CAS, zinc finger nuclease, meganuclease, or TALENs technology.

35. The method of paragraphs 28 through 34, wherein the one or more sry alleles are introduced into the genome using transposon systems, recombinant viral techniques, electroporation and microinjection of zygote pronuclei.

36. The method of paragraphs 28 through 35, wherein the somatic cell nucleus is implanted in an enucleated oocyte to provide a renucleated oocyte.

37. The method of paragraphs 28 through 36, wherein the renucleated oocyte is implanted in a surrogate mother.

38. The method of paragraphs 28 through 37, wherein the sry allele is under the control of its native promoter.

39. The method of paragraphs 28 through 38, wherein the sry allele is under the control of a non-native promoter.

40. The method of paragraphs 28 through 39, wherein the promoter is an inducible promoter or a constitutive promoter.

41. The method of paragraphs 28 through 40, wherein the inducible promoter comprises tetracycline, doxycycline, ecdysone, rapamycin, Hsp70.3, LAC or TRE.

42. The method of paragraphs 28 through 41, wherein the constitutive promoter comprises CMV, CaMV 35s, SV40, CMV, UBC, EF1A, PGK and CAGG.

43. The method of paragraphs 28 through 42, wherein the sry allele is injected into the pronucleus of a zygote.

44. The method of paragraphs 28 through 43, wherein the sry allele is injected into the cytoplasm of a zygote.

45. The method of paragraphs 28 through 44, wherein the allele is injected as RNA directly into a zygote using TALEN or ZFN RNA, CRISPR/CAS or meganuclease technology.

46. The method of paragraphs 28 through 45, wherein the non-human animal is a therian animal.

47. The method of paragraphs 21 through 46, wherein the sry allele is an ortholog.

48. The method of paragraph s 28 through 47, wherein the sry allele is a paralog.

49. A method for making a sterile, phenotypically female livestock having an XY genotype comprising; a genomic modification an HMG box of a sry allele.

50. The method of paragraphs 49, wherein the modification results in a break in protein synthesis of a gene.

51. The method of paragraphs 49 and 50, wherein the modification results in improper binding of the sry protein to its target gene.

52. The method of paragraphs 49 through 51, wherein the modification comprises and insert or a deletion.

53. The method of paragraphs 49 through 52, wherein the modification is made in a somatic cell or the nucleus of a zygote.

54. The method of paragraphs 49 through 53, wherein the somatic cell nucleus is transferred into an enucleated egg to provide a renuclated egg.

55. The method of paragraphs 49 through 54, wherein the renucleated egg is implanted into the uterus of a surrogate mother.

56. The method of paragraphs 49 through 55, wherein the genomic modification results from direct injection of specific nucleotides into a zygote.

57. The method of paragraphs 49 through 56, wherein the modification is carried out using precision gene editing using zinc finger nuclease, meganuclease, TALENs or CRISPR/CAS technology.

58. The method of paragraphs 49 through 57, wherein the modification is an insertion, deletion or single nucleotide polymorphism resulting from non-homologous end joining.

59. The non-human animal of claims 1 through 58, wherein the animal comprises an animal genetically modified to express one or more introduced sry alleles without the use of a selectable marker.

60. A non-human animal cell having in its genome an introduced nucleic acid comprising one or more sry alleles.

61. The animal cell of paragraph 60, wherein the animal has an X and a Y chromosome and is phenotypically male.

62. The animal cell of paragraphs 60 or 61, wherein the animal has two X chromosomes and is phenotypically male.

63. The animal cell of paragraphs 60 through 62, wherein the animal is sterile. 64. The animal cell of paragraphs 60 through 63, wherein at least one introduced sry allele is on one or more X chromosomes.

65. The animal cell of paragraphs 60 through 64, wherein at least one introduced sry allele is on one or more autosomes.

66. The animal cell of paragraphs 60 through 65, wherein the one or more introduced sry alleles are introduced by nonmeiotic introgression.

67. The animal cell of paragraphs 60 through 66, wherein the one or more sry alleles is introduced into the genome using a transposon system.

68. The animal cell of paragraphs 60 through 67, wherein the transposon system is Sleeping Beauty, Passport, Frog Prince, Tol2, or PiggyBac.

69. The animal cell of paragraphs 60 through 68 where the one or more sry alleles is introduced into the genome without the use of selectable markers.

70. The animal cell of paragraphs 60 through 69 wherein the non-human animal is coselected for transposon transposition and non-meiotic introgression.

71. The animal cell of paragraphs 60 through 70, wherein non-meiotic introgression interrupts the native sry allele.

72. The animal cell of paragraphs 60 through 71, wherein the introduced sry alleles are hereditable.

73. The animal cell of paragraphs 60 through 72, wherein the animal is a livestock animal cell.

74. The animal cell of paragraphs 60 through 73, wherein the cell is a primary cell, primary somatic cell or zygote.

75. The animal cell of paragraphs 60 through 74, wherein transposon transposition provides multiple insertions of the sry allele.

76. The animal cell of paragraphs 60 through 75, wherein the cell is a non-human cell.

77. The animal cell of any of paragraphs 60 through 76, wherein the cell is cotransformed by transposon transposition and non-meiotic introgression.

78. The animal cell of paragraphs 60 through 77, wherein the cell is a cow, pig, chicken, goat, sheep, dog, cat, rodent, deer, vole or rabbit cell.

79. The animal cell of paragraphs 60 through 78, wherein the introduced allele is an ortholog.

80. The animal cell of paragraphs 60 through 79, wherein the introduced allele is a paralog.

81. An animal cell comprising a genomic modification of a native sry allele.

82. The animal cell of paragraphs 60 through 81, wherein the genomic modification is a modification of the HMG box.

83. The animal cell of paragraphs 60 through 82, wherein the modification is made by non-meiotic introgression.

84. The animal cell of paragraphs 60 through 83, wherein the cell has an X and a Y chromosome.

85. The animal cell of paragraphs 60 through 84, wherein the cell is a primary cell, a primary somatic cell or a zygote.

86. The animal cell of paragraphs 60 through 85, wherein the animal cell is a therian cell.

87. The animal cell of paragraphs 60 through 86, wherein the modification is an insertion or deletion.

88. A non-human animal made from any of the cells or methods of any of the preceding paragraphs.

89. A process for making a non-human animal cell according to any of the preceding paragraphs wherein one or more exogenous sry alleles are introduced into the cells genome.

90. The process of paragraph 89, wherein the introduction is accomplished using zinc finger nuclease, meganuclease, TALENs or CRISPR/CAS technology.

91. The process of paragraph 89, wherein the introduction is accomplished using transposon technology.

92. The process of paragraphs 89 through 91, wherein the cell is a primary cell, primary somatic cell or zygote.

93. A cell derived from the process of any of paragraphs 89-92.

94. A non-human animal derived from the cell of paragraphs 93.

All patents, publications, and journal articles set forth herein are hereby incorporated by reference herein; in case of conflict, the instant specification is controlling.

While this invention has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

1. A non-human animal having in its genome exogenous nucleic acid comprising one or more sry alleles.

2. The non-human animal of claim 1, wherein the animal cell has an X and a Y chromosome and is phenotypically male.

3. The non-human animal of claim 1, wherein the animal has two X chromosomes and is phenotypically male.

4. The non-human animal of claim 1, wherein at least one exogenous sry allele is on one or more X chromosomes.

5. The non-human animal of claim 1, wherein at least one exogenous sry allele is on one or more autosomes.

6. The non-human animal of claim 1, wherein the one or more exogenous sry alleles are introduced by nonmeiotic introgression.

7. The non-human animal of claim 1, wherein the one or more exogenous sry alleles is introduced into the genome using a transposon system.

8. The non-human animal of claim 7, wherein the transposon system is Sleeping Beauty, Passport, Frog Prince, Tol2, or PiggyBac.

9. The non-human animal of claim 1, where the one or more exogenous sry alleles is introduced into the genome without the use of selectable markers.

10. The non-human animal of claim 1, wherein the animal is coselected for transposon transposition and non-meiotic introgression.

11. The non-human animal of claim 6, wherein non-meiotic introgression interrupts the native sry allele.

12. The non-human animal of claim 1, wherein the exogenous sry alleles are hereditable.

13. A non-human animal cell having in its genome exogenous nucleic acid comprising one or more sry alleles.

14. The non-human animal cell of claim 13, wherein at least one exogenous sry allele is on one or more autosomes.

15. The non-human animal cell of claim 13, wherein the one or more exogenous sry alleles are introduced by nonmeiotic introgression.

16. The non-human animal cell of claim 13, wherein the one or more sry alleles is introduced into the genome using a transposon system.

17. The non-human animal cell of claim 16, wherein the transposon system is Sleeping Beauty, Passport, Frog Prince, Tol2, or PiggyBac.

18. The non-human animal cell of claim 13, wherein the cell is coselected for transposon transposition and non-meiotic introgression.

19. The non-human animal cell of claim 13, wherein the cell is a primary cell, primary somatic cell or zygote.

20. An animal cloned from a cell of claim 13.

21. A method for providing a sterile, phenotypically male animal comprising:
integrating one or more exogenous sry alleles into the genome of an animal.

22. The method of claim 21, wherein the one or more exogenous sry alleles are hereditable.

23. The method of claim 21, wherein when the animal has an XX genotype, the animal is sterile and phenotypically male, when the animal has an XY genotype, the animal passes the introduced sry alleles to its offspring.

24. The method of claim 21, wherein the one or more sry alleles are introduced into one or more autosomes.

25. The method of claim 21, wherein the one or more sry alleles are integrated into the genome by nonmeiotic introgression.

26. The method of claim 21, wherein the one or more sry alleles are introduced into the genome using CRISPR/CAS, zinc finger nuclease, meganuclease, or TALENs technology.

27. The method of claim 21, wherein the one or more sry alleles are introduced into the genome using transposon systems comprising: Sleeping Beauty, Passport, Frog Prince, Tol2, or PiggyBac.

28. A cell derived from the method of claim 21.

29. A non-human animal derived from the cell of claim 28.
